(19) United States
(12) Patent Application Publication
(10) Pub. No.: US 2003/0175972 A1
(43) Pub. Date: Sep. 18, 2003

(54) Expression vectors and uses thereof

(76) Inventors: George Q. Daley, Weston, MA (US); Eugene Y. Koh, Cambridge, MA (US)

Correspondence Address: FISH & RICHARDSON PC, 225 FRANKLIN ST, BOSTON, MA, 02110, US

(21) Appl. No.: 09/982,223

(22) Filed: Oct. 18, 2001

Related U.S. Application Data
(60) Provisional application No. 60/241,879, filed on Oct. 20, 2000.

(51) Int. Cl. C12N 15/861; C12N 7/00; C12N 5/06

(52) U.S. Cl. 435/456; 435/325; 435/320.1; 435/235.1

(57) ABSTRACT

The invention includes viral vectors (e.g., retroviral vectors, e.g., replication deficient retroviral vectors), libraries comprising such vectors, retroviral particles produced by such vectors, retroviral packaging cell lines for production of these particles, integrated proviral sequences derived from the retroviral particles, circularized provirus sequences and mammalian cells upon which the provirus has been introduced. The invention also includes methods of using such sequence, vectors, particles and cells.

This application claims the benefit of a previously filed Provisional Application No. 60/241,879, filed Oct. 20, 2000, the contents of which is incorporated in its entirety. Work described herein was supported in part by funding from the National Institute of Health, Grant number 1R29CA76418-01.

### BACKGROUND OF THE INVENTION

The versatility of retroviral vectors as a method to express foreign genes in a variety of cells has made these transducing vectors widely used. Most of the interest has been motivated by the need to express known genes to examine their functional and biological roles. Retroviral vectors have been applied to introduce genes into numerous cell lines and in primary tissues leading to phenotypes ranging from simple drug resistance to more complex properties such as mimicking human leukemic disease (Daley et al. (1990) Science 247:824-830; Guild et al. (1988) J. Virol 62:3795-3801; Miller (1992) Curr. Topics MicroBiol Immunol. 158:1-24; Samarut et al. (1995) Methods Enzymol 254:206-228).

In the era of “discovery science,” however, the functional genomics efforts have moved away from examining one known gene at a time. Instead, the focus has become the ability to screen numerous genes, known and unknown, for a specific biological phenotype with the eventual hope of identifying novel genes and novel functions. Thus, the emphasis has shifted from creating a retroviral vector with one specific gene to generating cDNA libraries in retroviral vectors in order to express a panel of known and unknown genes. With the generation of retroviral cDNA libraries, the introduction of millions of independent cDNAs can now be accomplished.

In yeast, methods have been devised such that problems associated with gene isolation and discovery of gene function can be addressed in an efficient manner. For example, in yeast it is possible to isolate genes via their ability to complement specific phenotypes. In yeast, targeted insertional mutagenesis techniques can be sued to knock-out a gene's activity. However, methods for elucidation of mammalian gene function are lacking and can be inefficient.

### SUMMARY OF THE INVENTION

The invention is based, in part, on the development and characterization of expression vectors for phenotypic screens. These vectors provide: (1) high viral titers to facilitate screening of a large set of different cDNAs, (2) high levels of gene expression, (3) ease of recovery of the desired insert nucleic acid, and (4) the ability to screen libraries which include nucleic acids several kilobases in length. The recovery scheme can be PCR-based or the shuttle-based. With these improvements, the present vector system offers significant advantages and improvements over current retroviral expression cloning systems.

Accordingly, in one aspect, the invention features a nucleic acid. The nucleic comprises from 5′ to 3′: a) a packaging sequence; b) a heterologous insert sequence or restriction sites for insertion of a heterologous sequence; and c) a 3′ long terminal repeat (LTR) sequence, wherein at least two codons of the packaging sequence are altered so as to reduce formation of fusion polypeptides encoded by the packaging sequence or a portion thereof, and the heterologous insert sequence. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG codon of the naturally occurring packaging sequence and at least two internal ATG codons of the naturally occurring packaging sequence have been altered.

In one embodiment, the nucleic acid includes a gag packaging sequence, e.g., a gag packaging sequence which comprises the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the nucleic acid includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb, 400 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the nucleic acid includes at least one additional insert sequence, e.g., the nucleic can be polycistronic. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In another embodiment, the nucleic acid can further include a lethal stuffer fragment. In one embodiment, the lethal stuffer can be present in the nucleic acid such that insertion of the heterologous nucleic acid into the sequence replaces, or disrupts the sequence encoding the lethal stuffer fragment.

In another embodiment, the nucleic acid includes a bacterial replicon. The bacterial replicon includes a bacterial marker and an origin of replication (ori). In an embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the selectable bacterial markers described above. The bacterial replicon can include a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR can include one or more of a U3 region, a U5 region or a promoter containing portion thereof, an R region and a polyadenylation signal. In one embodiment, the 3 ′LTR of the nucleic acid includes a proviral recovery sequence. The proviral recovery sequence can be located within a portion of the 3 ′LTR which duplicates upon integration, e.g., duplicates such that the recovered provirus includes a 5′LTR, a 3 ′LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the nucleic acid includes a 5′ long terminal repeat (LTR). The 5′LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3 ′LTRs includes at least two, three, four or five rare cutter restriction sites. The rare cutter sequence (or sequences) can be located within a portion of the 3 ′LTR which duplicates upon integration, e.g., duplicates such that the provirus includes a 5 ′LTR, a 3′ LTR and a heterologous sequence between the LTRs.

In another embodiment, the nucleic acid includes: a 5′ LTR and a 3′ LTR; a 5′ LTR and a 3′ LTR having at least one rare cutter restriction site; a 5′ LTR having at least one rare cutter restriction site and a 3′ LTR; a 5′ LTR having at least one rare cutter restriction site and a 3′LTR having at least one rare cutter restriction sites, e.g., a rare cutter restriction site which is the same or a different rare cutter restriction site than in the 5′LTR; a 5′ LTR and a 3′LTR having at least one proviral recovery sequence; a 5′ LTR having at least one proviral recovery sequence and a 3′LTR; a 5′ LTR having at least one proviral recovery sequence and a 3′LTR having at least one proviral sequence; a 5′LTR having at least one proviral sequence and at least one rare cutter restriction site and a 3′LTR; a 5′ LTR and a 3′ LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′ LTR having at least one rare cutter restriction site and a 3′LTR having at least one proviral sequence; a 5′ LTR having at least one proviral sequence and a 3′LTR having at least one rare cutter restriction site; a 5′ LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′ LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

The nucleic acid can be linear or circular. The nucleic acid can be integrated in a chromosome, e.g., a mammalian chromosome, or a fragment. The nucleic acid can be packaged in a lipid bilayer having viral envelope polypeptides, e.g., a virion or retroviral particle.

In another aspect, the invention features a particle or retrovirus-like particle. The particle comprises a lipid bilayer having a viral envelope polypeptide disposed therein, and a nucleic acid disposed within. The nucleic acid comprises: a) a packaging sequence; b) a heterologous insert sequence; and c) a 3′LTR sequence, wherein at least two codons of the packaging sequence are altered so as to reduce formation of fusion polypeptides encoded by the packaging sequence or a portion thereof, and the heterologous insert sequence. The nucleic acid can be a ribonucleic acid or a deoxtribonucleic acid. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG codon of the naturally occurring packaging sequence and at least two internal ATG codons of the naturally occurring packaging sequence have been altered.

In one embodiment, the ribonucleic acid includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence corresponds to the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, heterologous insert sequence can be a sequence of interest, e.g., a polypeptide encoding sequence, a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the nucleic acid includes at least one additional insert sequence, e.g., the nucleic can be polycistronic. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In one embodiment, the nucleic acid includes a bacterial replicon. The bacterial replicon includes a bacterial marker and an origin of replication (ori). Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during amplification. For example, an origin of replication, which can be used in several bacterial species, is colEI. ColEI can be used, for example, in one or more of Bh5α, DH10B, JM109 and XL1blue. The bacterial marker can be any of the selectable bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR can include one or more of a U3 region, a U5 region or a promoter containing portion thereof, an R region and a polyadenylation signal. In one embodiment, the 3′ LTR of the nucleic acid includes a proviral recovery sequence. The proviral recovery sequence can be located within a portion of the 3′ LTR which duplicates upon integration, e.g., duplicates such that the recovered provirus includes a 5′ LTR, a 3′LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the nucleic acid includes a 5′ long terminal repeat (LTR). The 5′ LTR can include one or more of: a U5 region which includes a promoter (e.g., an internal LTR promoter or other inducible promoter), an R region, a U3 region, and a primer binding site. The 5′LTR can include a U3 region, an R region and a promoter-containing portion of a U5 region, in that order from 5′ to 3′.

In one embodiment, one or both of the 5′ and 3′LTRs further comprises at least one rare cutter restriction site (e.g., an 8-bp recognition site or larger). For example, the rare cutter restriction site can be a site for Not1, SfiI, PacI or P1-SceI. In another embodiment, one or both of the 5′ and 3′ LTRs includes at least two, three, four or five rare cutter restriction sites. The rare cutter sequence (or sequences) is located within a portion of the 3′ LTR which duplicates upon integration, e.g., duplicates such that the provirus includes a 5′ LTR, a 3′ LTR and a heterologous sequence between the LTRs.

In another embodiment, the nucleic acid includes: a 5′ LTR and a 3′ LTR; a 5′ LTR and a 3′ LTR having at least one rare cutter restriction site; a 5′LTR having at least one rare cutter restriction site and a 3′LTR; a 5′ LTR having at least one rare cutter restriction site and a 3′LTR having at least one rare cutter restriction sites, e.g., a rare cutter restriction site which is the same or a different rare cutter restriction site than in the 5′ LTR; a 5′LTR and a 3′LTR having at least one proviral recovery sequence; a 5′ LTR having at least one proviral recovery sequence and a 3′LTR; a 5′LTR having at least one proviral recovery sequence and a 3′LTR having at least one proviral sequence; a 5′LTR having at least one proviral sequence and at least one rare cutter restriction site and a 3′LTR; a 5′ LTR and a 3′ LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′ LTR having at least one rare cutter restriction site and a 3′LTR having at least one proviral sequence; a 5′ LTR having at least one proviral sequence and a 3′LTR having at least one rare cutter restriction site; a 5′LTR having at least one proviral sequence and a 3′ LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

In another aspect, the invention features a library of nucleic acids. Each nucleic acid of the library comprises: a) a packaging sequence; b) a heterologous insert sequence; and c) a 3′ long terminal repeat (LTR) sequence, wherein at least two codons of the packaging sequence are altered so as to reduce formation of fusion polypeptides encoded by the packaging sequence or a portion thereof, and the heterologous insert sequence. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG codon of the naturally occurring packaging sequence and at least two internal ATG codons of the naturally occurring packaging sequence have been altered.

In one embodiment, each of the nucleic acids includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, each of the nucleic acids can include a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, each of the nucleic acids includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, each of the nucleic acids includes both a mammalian marker sequence and a bacterial marker sequence.

In another embodiment, each of the nucleic acids includes at least one additional insert sequence, e.g., the nucleic can be polycistronic. For example, the nucleic acid can include a first insert sequence and a second insert sequence (e.g., the first sequence can be a sequence of interest and the second sequence can be a marker sequence). The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In one embodiment, the nucleic acid includes a bacterial replicon. The bacterial replicon includes a bacterial marker and an origin of replication (ori). In one embodiment, each nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during amplification. For example, an origin of replication, which can be used in several bacterial species, is colEI. ColEI can be used, for example, in one or more of Bh5α, DH10B, JM109 and XL1blue. The bacterial marker can be any of the selectable bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR can include one or more of a U3 region, a U5 region or a promoter containing portion thereof, an R region and a polyadenylation signal. In one embodiment, the 3′ LTR of each of the nucleic acids includes a proviral recovery sequence. The proviral recovery sequence can be located within a portion of the 3′ LTR which duplicates upon integration, e.g., duplicates such that the recovered provirus includes a 5′LTR, a 3′ LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, each of the nucleic acids includes a 5′ long terminal repeat (LTR). The 5′ LTR can include one or more of: a U5 region which includes a promoter (e.g., an internal LTR promoter or other inducible promoter), an R region, a U3 region, and a primer binding site. The 5′LTR can include a U3 region, an R region and a promoter-containing portion of a U5 region, in that order from 5′ to 3′.

In another embodiment, one or both of the 5′ and 3′LTRs further comprises at least one rare cutter restriction site (e.g., an 8-bp recognition site or larger). For example, the rare cutter restriction site can be a site for Not1, SfiI, PacI or P1-SceI. In another embodiment, one or both of the 5′ and 3′LTRs includes at least two, three, four or five rare cutter restriction sites. The rare cutter sequence (or sequences) can be located within a portion of the 3′ LTR which duplicates upon integration, e.g., duplicates such that the provirus includes a 5′LTR, a 3′ LTR and a heterologous sequence between the LTRs.

In another embodiment, each of the nucleic acids includes: a 5′ LTR and a 3′ LTR; a 5′ LTR and a 3′ LTR having at least one rare cutter restriction site; a 5′ LTR having at least one rare cutter restriction site and a 3′LTR; a 5′ LTR having at least one rare cutter restriction site and a 3′LTR having at least one rare cutter restriction sites, e.g., a rare cutter restriction site which is the same or a different rare cutter restriction site than in the 5′ LTR; a 5′LTR and a 3′LTR having at least one proviral recovery sequence; a 5′ LTR having at least one proviral recovery sequence and a 3′LTR; a 5′ LTR having at least one proviral recovery sequence and a 3′LTR having at least one proviral sequence; a 5′LTR having at least one proviral sequence and at least one rare cutter restriction site and a 3′LTR; a 5′ LTR and a 3′ LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′ LTR having at least one rare cutter restriction site and a 3′LTR having at least one proviral sequence; a 5′ LTR having at least one proviral sequence and a 3′LTR having at least one rare cutter restriction site; a 5′ LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′ LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

In one embodiment, each insert nucleic acid sequence in the library is unique. For example, each insert nucleic acid sequence can differ from all other insert nucleic acid sequences of the library by 1, or more nucleotide differences, (e.g., about 2, 3, 4, 5, 8, 16, 32, 64 or more differences; and, by way of example, has about 800, 256, 128, 64, or 32, 16, 8, 4, or fewer differences).

In one embodiment, the insert nucleic acids can be nucleic acids (e.g., an mRNA or cDNA) expressed in a tissue, e.g., a normal or diseased tissue. In another embodiment, the insert nucleic acids can encode mutants or variants of a scaffold protein (e.g., an antibody, zinc-finger, polypeptide hormone etc.). In yet another embodiment, the nucleic acids encode random amino acid sequences, patterned amino acids sequences, or designed amino acids sequences (e.g., sequence designed by manual, rational, or computer-aided approaches). The library of insert nucleic acid sequences can include a plurality from a first source, and plurality from a second source. For example, each plurality can be maintained in a separate container. Insert nucleic acids encoding polypeptides can be obtained from a collection of full-length expressed genes, a cDNA library, or a genomic library.

In another aspect, the invention features a packaging cell that comprises a viral envelope polypeptide and a nucleic acid as described herein. The nucleic acid can comprise: a) a packaging sequence; b) a heterologous insert sequence; and c) a 3′ long terminal repeat (LTR) sequence, wherein at least one, two or more codons of the packaging sequence are altered so as to reduce formation of fusion polypeptides encoded by the packaging sequence or a portion thereof, and the heterologous insert sequence. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG codon of the naturally occurring packaging sequence and at least two internal ATG codons of the naturally occurring packaging sequence have been altered.

In one embodiment, the packaging cell includes nucleic acid which includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the packaging cell includes a nucleic acid which includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the packaging cell includes nucleic acid which includes at least one additional insert sequence, e.g., the nucleic can be polycistronic, e.g., the nucleic acid can be dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In one embodiment, the packaging cell includes a nucleic acid which includes a bacterial replicon. The bacterial replicon includes a bacterial marker and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the selectable bacterial markers described above. In one embodiment, the bacterial replicon can include a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR of the nucleic acid can include one or more of a U3 region, a U5 region or a promoter containing portion thereof, an R region and a polyadenylation signal. In one embodiment, the 3′ LTR of the nucleic acid includes a proviral recovery sequence. The proviral recovery sequence can be located within a portion of the 3′ LTR which duplicates upon integration, e.g., duplicates such that the recovered provirus includes a 5′LTR, a 3′LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the packaging cell includes a nucleic acid which includes a 5′ long terminal repeat (LTR). The 5′ LTR can include one or more of: a U5 region which includes a promoter (e.g., an internal LTR promoter or other inducible promoter), an R region, a U3 region, and a primer binding site. The 5′LTR can include a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3′ LTRs includes at least two, three, four or five rare cutter restriction sites. The rare cutter sequence (or sequences) can be located within a portion of the 3′ LTR which duplicates upon integration, e.g., duplicates such that the provirus includes a 5′ LTR, a 3′LTR and a heterologous sequence between the LTRs.

In another embodiment, the packaging cell includes a nucleic acid which includes: a 5′ LTR and a 3′ LTR; a 5′ LTR and a 3′ LTR having at least one rare cutter restriction site; a 5′LTR having at least one rare cutter restriction site and a 3′ LTR; a 5′ LTR having at least one rare cutter restriction site and a 3′LTR having at least one rare cutter restriction sites, e.g., a rare cutter restriction site which is the same or a different rare cutter restriction site than in the 5′LTR; a 5′LTR and a 3′ LTR having at least one proviral recovery sequence; a 5′LTR having at least one proviral recovery sequence and a 3′LTR; a 5′ LTR having at least one proviral recovery sequence and a 3′LTR having at least one proviral sequence; a 5′LTR having at least one proviral sequence and at least one rare cutter restriction site and a 3′LTR; a 5′ LTR and a 3 LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′ LTR having at least one rare cutter restriction site and a 3′ LTR having at least one proviral sequence; a 5′ LTR having at least one proviral sequence and a 3′LTR having at least one rare cutter restriction site; a 5′ LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′ LTR having at least one rare cutter restriction site and a 3′LTR having at least one rare cutter restriction site and at least one proviral recovery sequence.

The 5′ LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

In another aspect, the invention features a mammalian cell that comprises a non-naturally occurring nucleic acid, e.g., a nucleic acid described herein. The non-naturally occurring nucleic acid comprises: a) a packaging sequence; b) a heterologous insert sequence; and c) a 3′ long terminal repeat (LTR) sequence, wherein at least one, two, codon(s) of the packaging sequence are altered so as to reduce formation of fusion polypeptides encoded by the packaging sequence or a portion thereof, and the heterologous insert sequence. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG initiation codon of the naturally occurring packaging sequence and at least two internal ATG codons of the naturally occurring packaging sequence have been altered.

In one embodiment, the cell includes a nucleic acid which includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the cell includes a nucleic acid which includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the cell includes a nucleic acid which includes both a mammalian marker sequence and a bacterial marker sequence.

In another embodiment, the cell includes a nucleic acid includes at least one additional insert sequence, e.g., the nucleic can be polycistronic, e.g., dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In one embodiment, the cell includes a nucleic acid which includes a bacterial replicon. The bacterial replicon includes a bacterial marker and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the selectable bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR of the nucleic acid can include one or more of a U3 region, a U5 region or a promoter containing portion thereof, an R region and a polyadenylation signal. In one embodiment, the 3′ LTR of the nucleic acid includes a proviral recovery sequence. The proviral recovery sequence can be located within a portion of the 3′ LTR which duplicates upon integration, e.g., duplicates such that the recovered provirus includes a 5′LTR, a 3′LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the cell includes a nucleic acid which includes a 5′ long terminal repeat (LTR). The 5′ LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3′ LTRs includes at least two, three, four or five rare cutter restriction sites. The rare cutter sequence (or sequences) can be located within a portion of the 3′ LTR which duplicates upon integration, e.g., duplicates such that the provirus includes a 5′ LTR, a 3′LTR and a heterologous sequence between the LTRs.

In another embodiment, the cell includes a nucleic acid which includes: a 5′ LTR and a 3′ LTR; a 5′ LTR and a 3′ LTR having at least one rare cutter restriction site; a 5′LTR having at least one rare cutter restriction site and a 3′LTR; a 5′ LTR having at least one rare cutter restriction site and a 3′LTR having at least one rare cutter restriction sites, e.g., a rare cutter restriction site which is the same or a different rare cutter restriction site than in the 5′LTR; a 5′LTR and a 3′LTR having at least one proviral recovery sequence; a 5′ LTR having at least one proviral recovery sequence and a 3′LTR; a 5′ LTR having at least one proviral recovery sequence and a 3′LTR having at least one proviral sequence; a 5′LTR having at least one proviral sequence and at least one rare cutter restriction site and a 3′LTR; a 5′ LTR and a 3′ LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′LTR having at least one rare cutter restriction site and a 3′LTR having at least one proviral sequence; a 5′ LTR having at least one proviral sequence and a 3′LTR having at least one rare cutter restriction site; a 5′ LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′ LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

In another aspect, the invention features a proviral sequence derived from a mammalian cell described herein. The proviral sequence comprises: a) a packaging sequence; b) a heterologous insert sequence; and c) a 3′ long terminal repeat (LTR) sequence, wherein at least one, two, codon(s) of the packaging sequence are altered so as to reduce formation of fusion polypeptides encoded by the packaging sequence or a portion thereof, and the heterologous insert sequence. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG initiation codon of the naturally occurring packaging sequence and at least two internal ATG codons of the naturally occurring packaging sequence have been altered.

In one embodiment, the proviral sequence includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1 589-1591 of SEQ ID NO:1. The ATG codon can be altered such that one, two or all of the nucleotides of the ATG codon(s) have been altered, e.g., substituted.

In one embodiment, the proviral sequence includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the proviral sequence includes both a mammalian marker sequence and a bacterial marker sequence.

In another embodiment, the proviral sequence includes at least one additional insert sequence, e.g., the nucleic can be polycistronic, e.g., dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In one embodiment, the proviral sequence includes a bacterial replicon. The bacterial replicon includes a bacterial marker and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the selectable bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR of the proviral sequence can include one or more of a U3 region, a U5 region or a promoter containing portion thereof, an R region and a polyadenylation signal. In one embodiment, the 3′ LTR of the nucleic acid includes a proviral recovery sequence. The proviral recovery sequence can be located within a portion of the 3′ LTR which duplicates upon integration, e.g., duplicates such that the recovered provirus includes a 5′LTR, a 3′LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the proviral sequence includes a 5′ long terminal repeat (LTR). The 5′ LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3′LTRs includes at least two, three, four or five rare cutter restriction sites. The rare cutter sequence (or sequences) can be located within a portion of the 3′ LTR which duplicates upon integration, e.g., duplicates such that the provirus includes a 5′ LTR, a 3′LTR and a heterologous sequence between the LTRs.

In another embodiment, the proviral sequence includes: a 5′ LTR and a 3′ LTR; a 5′LTR and a 3′ LTR having at least one rare cutter restriction site; a 5′LTR having at least one rare cutter restriction site and a 3′LTR; a 5′ LTR having at least one rare cutter restriction site and a 3′LTR having at least one rare cutter restriction sites, e.g., a rare cutter restriction site which is the same or a different rare cutter restriction site than in the 5′ LTR; a 5′LTR and a 3′ LTR having at least one proviral recovery sequence; a 5′ LTR having at least one proviral recovery sequence and a 3′LTR; a 5′ LTR having at least one proviral recovery sequence and a 3′LTR having at least one proviral sequence; a 5′LTR having at least one proviral sequence and at least one rare cutter restriction site and a 3′LTR; a 5′ LTR and a 3′ LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′ LTR having at least one rare cutter restriction site and a 3′LTR having at least one proviral sequence; a 5′ LTR having at least one proviral sequence and a 3′LTR having at least one rare cutter restriction site; a 5′LTR having at least one proviral sequence and a 3′ LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

The provirus sequences of the invention can be present in an integrated form within the genome of a recipient mammalian cell, or may be present in a free, circularized form.

In another aspect, the invention features a kit comprising a nucleic acid described herein. The kit includes a nucleic acid comprising a) a packaging sequence; b) a heterologous insert sequence or restriction sites for insertion of a heterologous sequence; and c) a 3′ long terminal repeat (LTR) sequence, wherein at least one, two, codon(s) of the naturally occurring packaging sequence are altered so as to reduce formation of fusion polypeptides encoded by the packaging sequence or a portion thereof, and the heterologous insert sequence. In one embodiment, at least two ATG codons of the packaging sequence have been altered from the naturally occurring packaging sequence, for example, the ATG initiation codon of the naturally occurring packaging sequence and at least one internal ATG codon of the naturally occurring packaging sequence have been altered. In another embodiment, the ATG initiation codon of the naturally occurring packaging sequence and at least two internal ATG codons of the naturally occurring packaging sequence have been altered.

In one embodiment, the nucleic acid includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the kit can include a nucleic acid which includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the nucleic acid includes at least one additional insert sequence, e.g., the nucleic can be polycistronic, e.g., the nucleic acid can be dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV. In another embodiment, the nucleic acid can include restriction sites for insertion of a nucleic acid. A nucleic acid which includes such restriction sites can further include a heterologous sequence, e.g., a heterologous sequence encoding, e.g., a polylinker, and/or a marker protein, e.g., a mammalian marker protein.

In another embodiment, the nucleic acid can further include a lethal stuffer fragment. In one embodiment, the lethal stuffer can be present in the nucleic acid such that insertion of the heterologous nucleic acid into the sequence replaces, or disrupts the sequence encoding the lethal stuffer fragment.

In one embodiment, the nucleic acid includes a bacterial replicon. The bacterial replicon includes a bacterial marker and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, HD10B, JM109 and XL1blue. The bacterial marker can be any of the selectable bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

The 3′LTR can include one or more of a U3 region, a U5 region or a promoter containing portion thereof, an R region and a polyadenylation signal. In one embodiment, the 3′ LTR of the nucleic acid includes a proviral recovery sequence. The proviral recovery sequence can be located within a portion of the 3′ LTR which duplicates upon integration, e.g., duplicates such that the recovered provirus includes a 5′ LTR, a 3′ LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the nucleic acid includes a 5′ long terminal repeat (LTR). The 5′ LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3′ LTRs includes at least two, three, four or five rare cutter restriction sites. The rare cutter sequence (or sequences) can be located within a portion of the 3′ LTR which duplicates upon integration, e.g., duplicates such that the provirus includes a 5′ LTR, a 3′LTR and a heterologous sequence between the LTRs.

In another embodiment, the nucleic acid includes: a 5′ LTR and a 3′ LTR; a 5′ LTR and a 3′ LTR having at least one rare cutter restriction site; a 5′LTR having at least one rare cutter restriction site and a 3′LTR; a 5′ LTR having at least one rare cutter restriction site and a 3′LTR having at least one rare cutter restriction sites, e.g., a rare cutter restriction site which is the same or a different rare cutter restriction site than in the 5′ LTR; a 5′LTR and a 3′LTR having at least one proviral recovery sequence; a 5′ LTR having at least one proviral recovery sequence and a 3′LTR; a 5′ LTR having at least one proviral recovery sequence and a 3′LTR having at least one proviral sequence; a 5′LTR having at least one proviral sequence and at least one rare cutter restriction site and a 3′ LTR; a 5′ LTR and a 3′ LTR having at least one proviral sequence and at least one rare cutter restriction site; a 5′LTR having at least one proviral sequence and at least one rare cutter restriction site and a 3′ LTR having at least one proviral sequence and at least one rare cutter restriction site; a 5′ LTR having at least one rare cutter restriction site and a 3′LTR having at least one proviral sequence; a 5′ LTR having at least one proviral sequence and a 3′LTR having at least one rare cutter restriction site; a 5′LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′ LTR having at least one rare cutter restriction site and at least one proviral recovery sequence and a 3′LTR having at least one rare cutter restriction site; a 5′LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

The kit can further include: nucleic acid for recovery, packaging cell line, bacterial strain for recovery, bacterial strain for counter selection of vector (in some embodiments), wild-type virus, primers for amplification, control virus, control nucleic acid, and/or instructions. In one embodiment, the kit also includes a recombinase, a ligase, and/or a restriction endonuclease. For example, the recombinase can mediate recombination, e.g., site-specific recombination or homologous recombination, between a recombination site on the test nucleic acid and a recombination sequence on the vector nucleic acid. For example, the recombinase can be lambda integrase, HIV integrase, Cre, or FLP recombinase.

In another aspect, the invention features a nucleic acid which comprises from 5′ to 3′: a) a packaging sequence, wherein at least one ATG codon of the packaging sequence has been altered; b) a heterologous insert sequence or restriction sites for insertion of a heterologous sequence; and c) a 3′ LTR sequence, wherein the 3′ LTR comprises a proviral recovery sequence.

In one embodiment, one or more of the ATG initiation codon of the naturally occurring packaging sequence and an internal ATG codon of the naturally occurring packaging sequence have been altered. In another embodiment, the ATG initiation codon of the naturally occurring packaging sequence and at least one or two internal ATG codons of the naturally occurring packaging sequence have been altered.

In one embodiment, the nucleic acid includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the nucleic acid includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell. In another embodiment, the nucleic acid includes a bacterial selectable marker. Selectable bacterial markers can include, but are not limited to, kanamycin/G418, zeocin, actinomycin, ampicillin, gentamycin, tetracycline, chloramphenicol and penicillin resistance markers. The bacterial marker can be about 600 kb, 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the nucleic acid includes at least one additional insert sequence, e.g., the nucleic can be polycistronic, e.g., the nucleic acid can be dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In another embodiment, the nucleic acid can further include a lethal stuffer fragment. In one embodiment, the lethal stuffer can be present in the nucleic acid such that insertion of the heterologous nucleic acid into the sequence replaces, or disrupts the sequence encoding the lethal stuffer fragment.

In one embodiment, the nucleic acid includes a bacterial replicon. The bacterial replicon includes a bacterial marker and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the selectable bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

In one embodiment, the 3′ LTR of the nucleic acid includes a proviral recovery sequence which is located within a portion of the 3′ LTR which duplicates upon integration, e.g., duplicates such that the recovered provirus includes a 5′LTR, a 3′ LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the nucleic acid includes a 5′ long terminal repeat (LTR). The 5′ LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3′LTRs includes at least two, three, four or five rare cutter restriction sites. The rare cutter restriction site can be located within a portion of the 3′ LTR which duplicates upon integration, e.g., duplicates such that the recovered provirus includes a 5′LTR, a 3′ LTR and a heterologous sequence between the two LTRs. For example, the proviral recovery sequence can be in the U3 region of the 3′ LTR. This can result in a provirus which is flanked by rare cutter restriction sites.

In another embodiment, the nucleic acid includes: a 5′ LTR and a 3′ LTR having at least one rare cutter restriction site; a 5′ LTR having at least one rare cutter restriction site and a 3′LTR having at least one rare cutter restriction sites, e.g., a rare cutter restriction site which is the same or a different rare cutter restriction site than in the 5′ LTR; a 5′ LTR having at least one proviral recovery sequence and a 3′LTR having at least one proviral sequence; a 5′LTR having at least one proviral sequence and at least one rare cutter restriction site and a 3′ LTR having at least one proviral sequence and at least one rare cutter restriction site; a 5′LTR having at least one proviral sequence and at least one rare cutter restriction site and a 3′ LTR having at least one proviral sequence; a 5′ LTR having at least one rare cutter restriction site and a 3′LTR having at least one proviral sequence; a 5′ LTR having at least one proviral sequence and a 3′LTR having at least one rare cutter restriction site; a 5′ LTR having at least one proviral sequence and a 3′ LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′ LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

The nucleic acid can be linear or circular. The nucleic acid can be integrated in a chromosome, e.g., a mammalian chromosome, or a fragment. The nucleic acid can be packaged in a lipid bilayer having viral envelope polypeptides, e.g., a virion or retroviral particle.

In another aspect, the invention features a nucleic acid which comprises from 5′ to 3′: a) a packaging sequence, wherein at least one ATG codon of the naturally occurring packaging sequence has been altered; b) a heterologous insert sequence or restriction sites for insertion of a heterologous sequence; c) a bacterial marker sequence, wherein the bacterial marker is less than 600 basepairs in length; and d) a 3′ LTR sequence, wherein the 3′ LTR comprises a proviral recovery sequence.

In one embodiment, one or more of the ATG initiation codon of the naturally occurring packaging sequence and an internal ATG codon of the naturally occurring packaging sequence have been altered. In another embodiment, the ATG initiation codon of the naturally occurring packaging sequence and at least one or two internal ATG codons of the naturally occurring packaging sequence have been altered.

In one embodiment, the nucleic acid includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the nucleic acid includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell.

In another embodiment, the bacterial marker is about 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the nucleic acid includes at least one additional insert sequence, e.g., the nucleic can be polycistronic, e.g., the nucleic acid can be dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In another embodiment, the nucleic acid can further include a lethal stuffer fragment. In one embodiment, the lethal stuffer can be present in the nucleic acid such that insertion of the heterologous nucleic acid into the sequence replaces, or disrupts the sequence encoding the lethal stuffer fragment.

In one embodiment, the nucleic acid includes a bacterial replicon. The bacterial replicon includes the bacterial marker sequence and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

In one embodiment, the 3′ LTR of the nucleic acid includes a proviral recovery sequence which is located within a portion of the 3′ LTR which duplicates upon integration, e.g., duplicates such that the recovered provirus includes a 5′ LTR, a 3′ LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the nucleic acid includes a 5′ long terminal repeat (LTR). The 5′LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or PI-SceI. In another embodiment, one or both of the 5′ and 3′LTRs includes at least two, three, four or five rare cutter restriction sites. The rare cutter restriction site can be located within a portion of the 3′ LTR which duplicates upon integration, e.g., duplicates such that the recovered provirus includes a 5′LTR, a 3′ LTR and a heterologous sequence between the two LTRs. For example, the proviral recovery sequence can be in the U3 region of the 3′ LTR. This can result in a provirus which is flanked by rare cutter restriction sites.

In another embodiment, the nucleic acid includes: a 5′ LTR and a 3′ LTR having at least one rare cutter restriction site; a 5′ LTR having at least one rare cutter restriction site and a 3′LTR having at least one rare cutter restriction sites, e.g., a rare cutter restriction site which is the same or a different rare cutter restriction site than in the 5′LTR; a 5′ LTR having at least one proviral recovery sequence and a 3′LTR having at least one proviral sequence; a 5′LTR having at least one proviral sequence and at least one rare cutter restriction site and a 3′ LTR having at least one proviral sequence and at least one rare cutter restriction site; a 5′LTR having at least one proviral sequence and at least one rare cutter restriction site and a 3′ LTR having at least one proviral sequence; a 5′ LTR having at least one rare cutter restriction site and a 3′LTR having at least one proviral sequence; a 5′ LTR having at least one proviral sequence and a 3′LTR having at least one rare cutter restriction site; a 5′ LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′ LTR having at least one rare cutter restriction site and a 3′ LTR having at least one rare cutter restriction site and at least one proviral recovery sequence.

The 5′LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

The nucleic acid can be linear or circular. The nucleic acid can be integrated in a chromosome, e.g., a mammalian chromosome, or a fragment. The nucleic acid can be packaged in a lipid bilayer having viral envelope polypeptides, e.g., a virion or retroviral particle.

In another aspect, the invention features a nucleic acid which comprises: a) a packaging sequence; b) a heterologous insert sequence; c) a bacterial marker sequence, wherein the bacterial marker sequence is less than 600 basepairs in length; d) a 3′ LTR comprising a proviral recovery sequence, wherein the vector comprises and can express a heterologous insert sequence greater than about 8 kilobases in length.

In one embodiment, the packaging sequence includes at least on ATG codon which has been altered from the naturally occurring packaging sequence, e.g., one or more of the ATG initiation codon of the naturally occurring packaging sequence and an internal ATG codon of the naturally occurring packaging sequence have been altered. In another embodiment, the ATG initiation codon of the naturally occurring packaging sequence and at least one or two internal ATG codons of the naturally occurring packaging sequence have been altered.

In one embodiment, the nucleic acid includes a gag packaging sequence, e.g., a gag packaging sequence which includes the initiation codon of the gag coding sequence. For example, the gag sequence is an amino-terminal portion of the gag gene, e.g., a sequence of about 300 to 1500, or 500 to 1200, or 900 to 1100 nucleotides. In one embodiment, the gag sequence comprises the nucleotide sequence of SEQ ID NO:2, or a portion thereof. In another embodiment, the internal codon which is altered can be, for example: the codon at residues 1097-1099 of SEQ ID NO:1 and/or the codon at residues 1589-1591 of SEQ ID NO:1. The ATG codon can be altered such that one, two or all of the nucleotides of the ATG codon(s) have been altered, e.g., substituted.

In one embodiment, the nucleic acid includes a heterologous insert sequence. The insert sequence can be a sequence of interest, e.g., a polypeptide encoding sequence (e.g., a cDNA, full-length cDNA or genomic DNA), a nucleic acid encoding a ribozyme, a nucleic acid aptmer, a polylinker, and/or a marker protein, e.g., a mammalian marker protein. The marker can be a selectable, counter-selectable, or detectable marker. For example, mammalian selectable markers can include, but are not limited to, kanamycin/G418, hygromycib B or mycophenolic acid resistance markers. Detectable markers can include, but are not limited, a fluorescent marker (e.g., green fluorescent protein, variants thereof, red fluorescent protein, variants thereof, and the like) or a marker which can alter the fluorescence of a cell.

In another embodiment, the bacterial marker is about 550 kb, 500 kb, 450 kb or less in size. The bacterial marker can also include a bacterial promoter, e.g., an Em7 promoter. In one embodiment, the bacterial marker is a bleomycin gene or fragments or mutants thereof.

In one embodiment, the nucleic acid includes both a mammalian marker sequence and a bacterial marker sequence.

In another embodiment, the nucleic acid includes at least one additional insert sequence, e.g., the nucleic can be polycistronic, e.g., the nucleic acid can be dicistronic, tricistronic, etc. For example, the nucleic acid can include a first insert sequence and a second insert sequence. The first and second insert sequences can be under the control of the same or different promoters. When the insert sequences are under the control of the same promoter, an internal ribosomal entry site (IRES) sequence can be positioned between the first and second insert sequence. The IRES sequence can include, for example, IRES derived from foot and mouth disease (FDV), encephalomyocarditis virus, poliovirus and RDV.

In another embodiment, the nucleic acid can further include a lethal stuffer fragment. In one embodiment, the lethal stuffer can be present in the nucleic acid such that insertion of the heterologous nucleic acid into the sequence replaces, or disrupts the sequence encoding the lethal stuffer fragment.

In one embodiment, the nucleic acid includes a bacterial replicon. The bacterial replicon includes the bacterial marker sequence and an origin of replication (ori). In one embodiment, the nucleic acid includes only one origin of replication. Examples of suitable bacterial origins of replication include pUC, colEI, pSC101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue. The bacterial marker can be any of the bacterial markers described above. In one embodiment, the bacterial replicon includes a bacterial promoter, a bacterial marker and an origin of replication, and is less than 2 kb, 1.8 kb, 1.6 kb, 1.4 kb 1.2 kb, 1 kb in size. For example, the bacterial replicon can include an EM7 promoter, a gene encoding bleomycin or mutants and fragments thereof, and a col1EI origin of replication.

In one embodiment, the 3′ LTR of the nucleic acid includes a proviral recovery sequence which is located within a portion of the 3′ LTR which duplicates upon integration, e.g., duplicates such that the recovered provirus includes a 5′ LTR, a 3′ LTR and a heterologous sequence between the two LTRs. For example, the proviral recovery sequence can be in the U3 region of the 3′ LTR. In one embodiment, the proviral recovery sequence includes a recombinase site. This can result in a provirus which is flanked by recombinase sites. In one embodiment, the proviral recovery sequence comprises a nucleotide sequence which is specifically recognized by a recombinase enzyme. A recombinase enzyme can be used to cleave a nucleic acid sequence at its site of recognition in such a manner that excision via recombinase action leads to circularization of the excised nucleic acid. In one embodiment, the proviral recovery sequence includes a loxP recombination site or a mutant loxP recombination site, which is cleavable by a Cre recombinase enzyme. In another embodiment, the proviral recovery sequence includes an frt recombination site, which is cleavable by an flp recombinase enzyme.

In another embodiment, the nucleic acid includes a 5′ long terminal repeat (LTR). The 5′ LTR can include one or more of: a U5 region which includes a promoter (e.g., an internal LTR promoter or other inducible promoter), an R region, a U3 region, and a primer binding site. In one embodiment, the 5′LTR includes a U3 region, an R region and a promoter-containing portion of a U5 region, in that order from 5′ to 3′.

In one embodiment, one or both of the 5′ and 3′LTRs includes at least one rare cutter restriction site (e.g., an 8-bp recognition site or larger). For example, the rare cutter restriction site can be a site for Not1, SfiI, PacI or P1-SceI. In another embodiment, one or both of the 5′ and 3′LTRs includes at least two, three, four or five rare cutter restriction sites. The rare cutter restriction site can be located within a portion of the 3′ LTR which duplicates upon integration, e.g., duplicates such that the recovered provirus includes a 5′LTR, a 3′ LTR and a heterologous sequence between the two LTRs. For example, the proviral recovery sequence can be in the U3 region of the 3′ LTR. This can result in a provirus which is flanked by rare cutter restriction sites.

In another embodiment, the nucleic acid includes: a 5′LTR and a 3′ LTR having at least one rare cutter restriction site; a 5′ LTR having at least one rare cutter restriction site and a 3′LTR having at least one rare cutter restriction sites, e.g., a rare cutter restriction site which is the same or a different rare cutter restriction site than in the 5′ LTR; a 5′ LTR having at least one proviral recovery sequence and a 3′LTR having at least one proviral sequence; a 5′LTR having at least one proviral sequence and at least one rare cutter restriction site and a 3′ LTR having at least one proviral sequence and at least one rare cutter restriction site; a 5′LTR having at least one proviral sequence and at least one rare cutter restriction site and a 3′ LTR having at least one proviral sequence; a 5′ LTR having at least one rare cutter restriction site and a 3′LTR having at least one proviral sequence; a 5′ LTR having at least one proviral sequence and a 3′ LTR having at least one rare cutter restriction site; a 5′ LTR having at least one proviral sequence and a 3′LTR having at least one rare cutter restriction site and at least one proviral sequence; a 5′ LTR having at least one rare cutter restriction site and at least one proviral sequence and a 3′ LTR having at least one proviral recovery sequence; a 5′LTR having at least one rare cutter restriction site and at least one proviral recovery sequence and a 3′ LTR having at least one rare cutter restriction site; a 5′LTR having at least one rare cutter restriction site and a 3′ LTR having at least one rare cutter restriction site and at least one proviral recovery sequence.

The 5′ LTR, 3′ LTR or both can be from a retrovirus, e.g., Moloney murine leukemia virus (MoMLV); mouse mammary tumor virus (MMLV); murine stem cell virus (MSCV); Rous Sarcoma virus (RSV); feline leukemia virus (FLV); bovine leukemia virus; spuma virus; a lentivirus (e.g., human immunodeficiency virus (HIV-1), and simian immunodeficiency virus (SIV)).

The nucleic acid can be linear or circular. The nucleic acid can be integrated in a chromosome, e.g., a mammalian chromosome, or a fragment. The nucleic acid can be packaged in a lipid bilayer having viral envelope polypeptides, e.g., a virion or retroviral particle. In one embodiment, the heterologous insert sequence can be a sequence of interest, e.g., a polypeptide encoding sequence (e.g., a cDNA, full-length cDNA or genomic DNA), a nucleic acid encoding a ribozyme, etc., which is at least 8, 8.5, 9, 9.5, 10, 10.5, 11, 11.15, 12 kilobases in length.

In another aspect, the invention features a method of generating a library. The method comprises: (1) providing an insert nucleic acid library (e.g., a cDNA library); (2) inserting at least a portion (i.e., a sub-library) of the nucleic acids from the library into a nucleic acid vector described herein. The method can also include introducing the sub-library into mammalian cells, e.g., cells of a packaging cell line. The cell can be adapted to expresss a retroviral envelope (env) protein and/or a retroviral reverse transcriptase (pol). Preferably, the cell is unable to produce a wildtype retrovirus, e.g., the cell lacks a gene encoding a gag polypeptide. The method can also include harvesting retroviral particles containing a nucleic acid as described herein.

In one embodiment, the method of generating the library further includes separating the insert nucleic acids into at least two sub-libraries prior to insertion of the nucleic acids into a vector, and then inserting each of the sub-libraries into a nucleic acid vector described herein. The nucleic acid library can be separated into sub-libraries based upon the size of the insert nucleic acid. By separating based upon size, preferential amplification of smaller nucleic acids can be reduced. For example, the nucleic acid library can be separated into sub-libraries having insert nucleic acids of about 1 kb or less, and those with insert nucleic acids greater than about 1 kb. In another embodiment, the nucleic acid library is separated into at least three sub-libraries: insert nucleic acids of about 500 basepairs or less, insert nucleic acids of about 1 to 3 kb, and insert nucleic acids greater than about 3 kb. The nucleic acid library can be subjected to size fractionalization, e.g., using SDS-PAGE, and separated based upon size into at least two, three, four sub-libraries.

The library generated can be: a normalized or non-normalized library for sense or antisense expression; a library selected against a specific chromosome or region of a chromosome (e.g., YACs); a library generated from any tissue source, e.g., from healthy or diseased tissue.

In another aspect, the invention features a method that comprises: (1) introducing a first nucleic acid, e.g., a nucleic acid described herein, into a packaging cell; (2) harvesting a particle from the cell; and (3) contacting the particle to a target cell.

The particle is a lipid bilayer having a retroviral envelope protein disposed therein, and a particle nucleic acid that includes the first nucleic acid or a copy thereof, e.g., an RNA copy thereof.

The packaging cell can be a cell of a packaging cell line. The packaging cell can be adapted to expresss a retroviral envelope (env) protein and/or a retroviral reverse transcriptase (pol). Preferably, the cell is unable to produce a wildtype retrovirus, e.g., the cell lacks a gene encoding a gag polypeptide.

The method can further comprise one or more of: (4) expressing an insert nucleic acid sequence that is included in the first nucleic acid, e.g., a nucleic acid described herein; (5) integrating the first nucleic acid, e.g., a nucleic acid described herein, into a chromosome of the target cell; (6) detecting a parameter of the target cell, e.g., by detecting a parameter of the cell by any screening method described herein, e.g., detecting information about the abundance, modification or activity of expressed polypeptides, the abundance of the expressed nucleic acids, and.or the abundance or modification state of metabolites; (7) infecting the target cell with a replication competent virus; (8) recovering a region of interest of the first nucleic acid from the target cell, e.g., by the PCR-mediated, restriction enzyme or cre-mediated recovery methods described herein; and (9) excising a region of interest of the first nucleic acid from the target cell, e.g., by reversion, e.g., by the reversion method described herein.

In one embodiment, the method includes detecting a parameter of the target cell by one or more of: detecting survival or proliferation advantage or disadvantage, activation or inactivation of a signal pathway, expression levels of a marker sequence, presence or absence of a cell function or characteristic.

In another aspect, the invention features a method that comprises (1) contacting a particle described herein to a target cell.

The method can further comprise one or more of: (2) expressing a nucleic acid sequence that is included in the first nucleic acid, e.g., a nucleic acid described herein; (3) integrating the first nucleic acid, e.g., a nucleic acid described herein, into a chromosome of the target cell; (4) detecting a parameter of the target cell e.g., by detecting a parameter of the cell by any screening method described herein (e.g., by detecting survival or proliferation advantage or disadvantage, activation or inactivation of a signal pathway, expression levels of a marker sequence, presence or absence of a cell function or characteristic; (5) infecting the target cell with a replication competent virus; (6) recovering a region of interest of the first nucleic acid from the target cell, e.g., by the PCR-mediated, restriction enzyme or cre-mediated recovery methods described herein; and (7) excising a region of interest of the first nucleic acid from the target cell, e.g., by reversion, e.g., by the reversion method described herein.

In one embodiment, multiple parameters of the target cell are detected. For example, the parameters can include information about the abundance, modification, and/or activity of expressed polypeptides (e.g., the proteome), the abundance of expressed nucleic acids (e.g., the transcriptosome), and/or the abundance and/or modification state of metabolites (e.g., the metabolome).

In another aspect, the invention features a method of identifying a sequence of interest. The method comprises (1) contacting a library of particles described herein to target cells; and (2) identifying a target cell, e.g., based upon the screening methods described herein.

### BRIEF DESCRIPTION OF THE DRAWINGS

FIG. 1 is a nucleotide sequence of a gag packaging sequence having an initiation ATG codon which has been altered. (SEQ ID NO:1)

FIG. 2 is a nucleotide sequence of a gag packaging sequence having the initiation codon and two internal ATG codons at residues which have been altered. (SEQ ID NO:2)

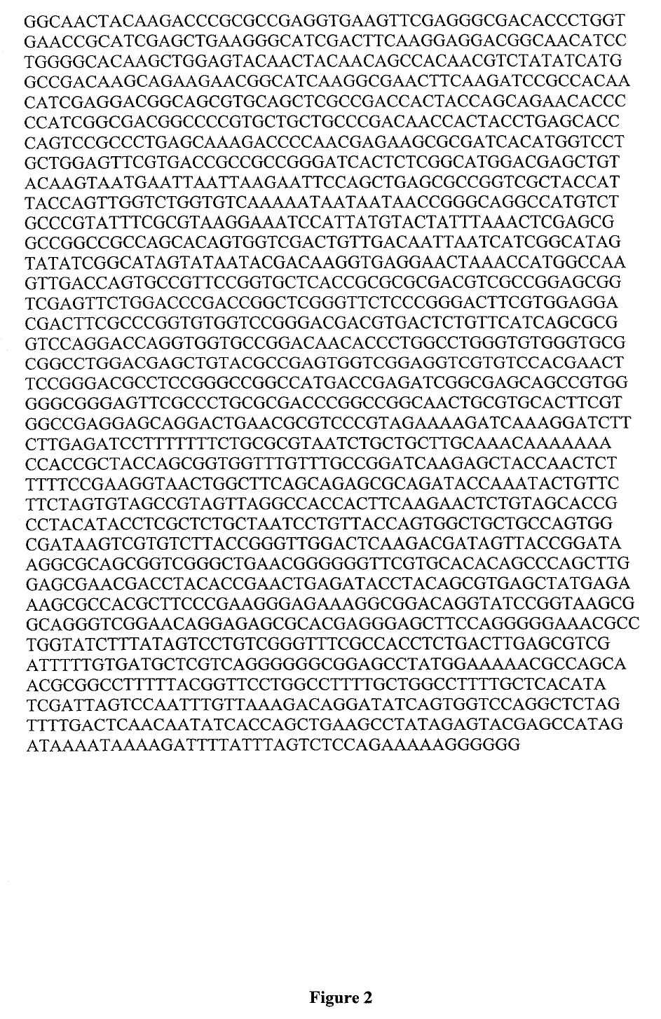

FIG. 3 is an alignment between an amino-terminal portion of the gag gene (a portion of SEQ ID NO:1) and an amino-terminal portion of the gag gene in which potential initiation codons have been altered (a portion of SEQ ID NO:2) to reduce formation of fusion polypeptides encoded by the packaging sequence or portions thereof and a heterologous insert sequence.

FIG. 4 depicts pEYK retroviral vector systems. The pEYK vectors originated from the pMX vector. The lines at the end of each provirus (except for pEYK3. l) designate an amp-ColE bacterial plasmid backbone. The stars (**) denote the mutagenized gag region. LTR denotes long terminal repeats, GFP denotes a green fluorescent protein encoding sequence, ble denotes a bleomycin encoding sequence, the loxP arrow denotes a loxP recombination sequence (the tip of the arrow indicating where Cre recombinase cleaves the sequence)

FIG. 5 depicts the generation of pEYK2 retroviral vector. FIG. 5A depicts the pMX vector which contains an extended gag region and a lethal stuffer sequence. FIG. 5B depicts the pEYK2 vector in which two-rounds of site-directed mutagenesis were performed on pMX to alter to internal ATG codons (residues 1355 and 1847) of the gag packaging sequence.

FIG. 6 depicts the generation of an LTR which includes a proviral recovery sequence and rare cutter restriction sites (also referred to herein as a “959 LTR”). The 959 LTR was created to generate an integrated provirus with flanking restriction enzyme sites (NotI, Pac I, AscI) and loxP sites. An oligonucleotide sequence containing the NotI, LoxP, PacI and AscI sites was placed at the NheI site in the U3 region of the LTR. The pEYK7 vector contains a single LTR, provides a source of the 959LTR and serves as an acceptor plasmid for rescue of the pEYK2.1 vector.

FIG. 7 depicts the use of a 3′ 959 LTR to obtain duplication of this site in the 5′LTR of the integrated provirus. The 959 LTR uses the life cycle of the retrovirus to copy restriction enzyme sites (NotI, PacI and AscI) and the loxP site into both the 5′ and 3′LTRs of the integrated provirus.

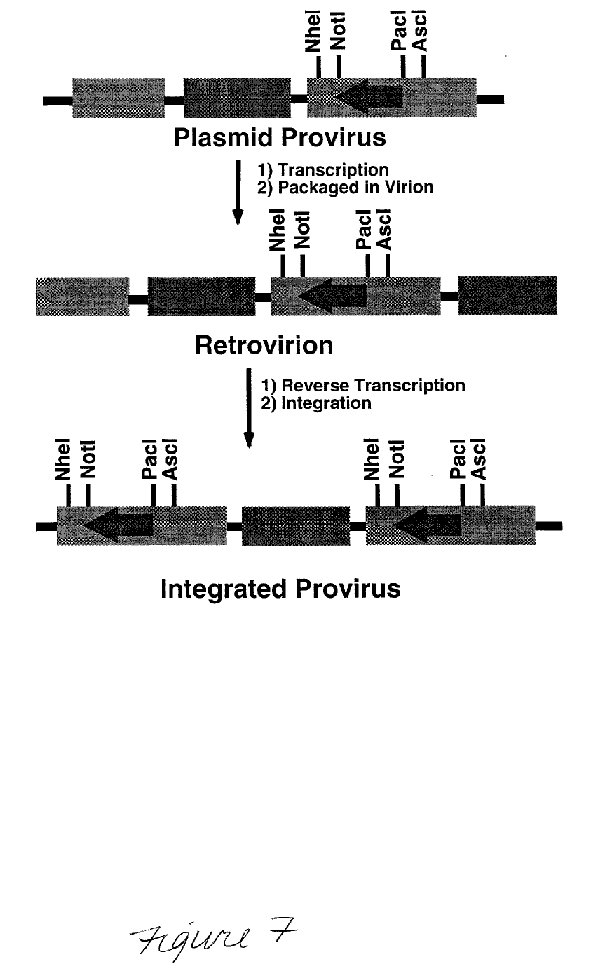

FIG. 8 depicts the cloning strategy for pEYK3.1 retroviral vector. PDSL was generated from pDOL by digesting with XbaI and self ligation. The SV40-noeR-pBRori fragment of pDSL was replaced with the EM7-ble-colEI fragment to generate pZSL vector. GFP-3M was inserted between the SalI and BamHI site to create the pGZSL vector. The 959 LTR from pEYK7 was cloned into the NheI and Kpn I sites of pGZSL, resulting in the pEYK3 vector. The packaging signal and mutagenized 1 kb gag region of pEYK2 was placed into pEYK3 via the KpnI and BamHI sited, yielding pEYK3.1.

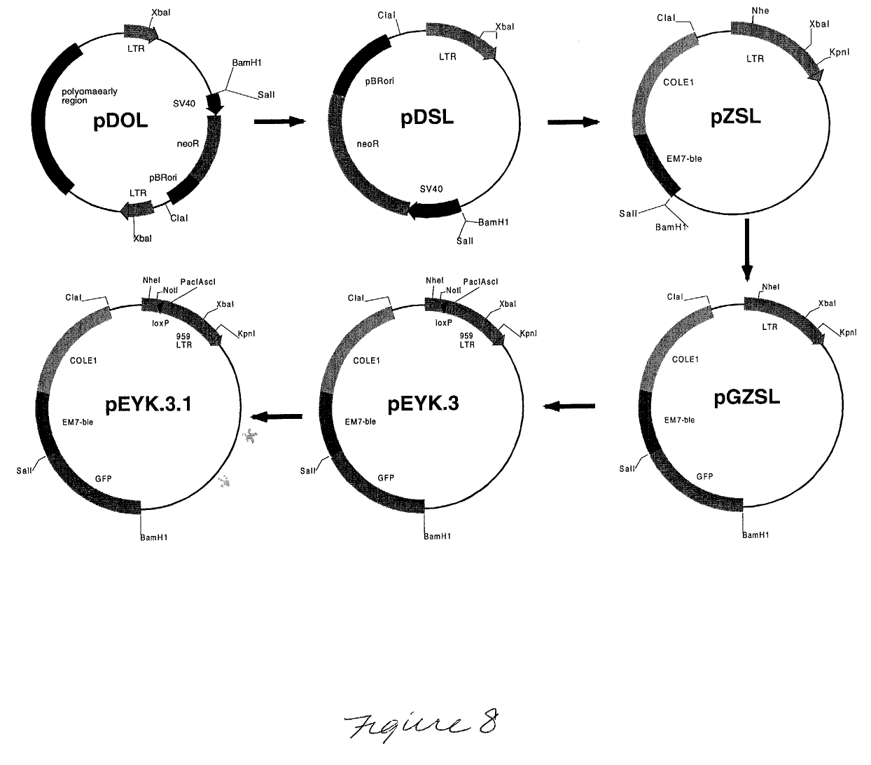

FIG. 9 shows GFP fluorescence of pEYK2.2 and pEYK2.3 by FACS analysis. Retorviral supemants (50 μL) were used to infect 1×106 BaF/3 cells. Two days-post infection, FACS analysis revealed that the modified LTR does not affect expression levels or retroviral titers.

FIG. 10 shows GFP fluorescent levels of pEYK3 (which does not contain the mutagenized gag sequence) and pEYK3.1 (which does contain the mutagenized gag sequence). The shuttle vectors pEYK3 and pEYK3.1 were analyzed for expression levels and titers. The pEYK3.1 retroviral construct containing the mutated gag sequence showed four-fold higher expression of GFP as measured by fluorescence when compared to pEYK3 parental vector.

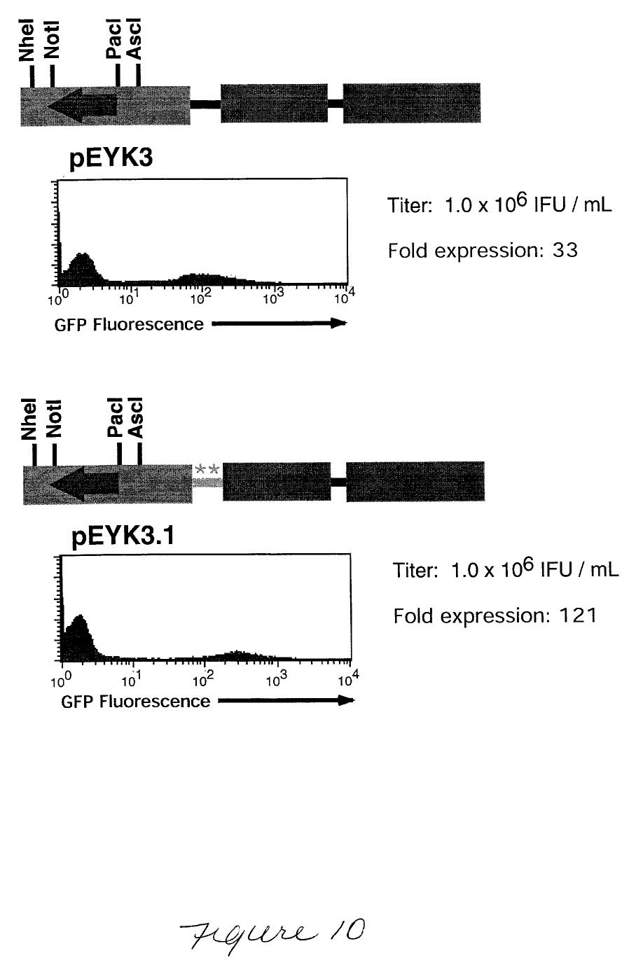

FIG. 11 depicts a recovery strategy using the pEYK2. 1 retroviral vector. To recover the integrated provirus from genomic DNA, restriction enzyme digestions with either NotI or PacI or AscI are preformed. The resulting genomic fragments are ligated into the pEYK7 acceptor vector plasmid, resulting in a reconstituted virus that can be selected and amplified in the presence of both ampicillin (amp) and zeocin (ble).

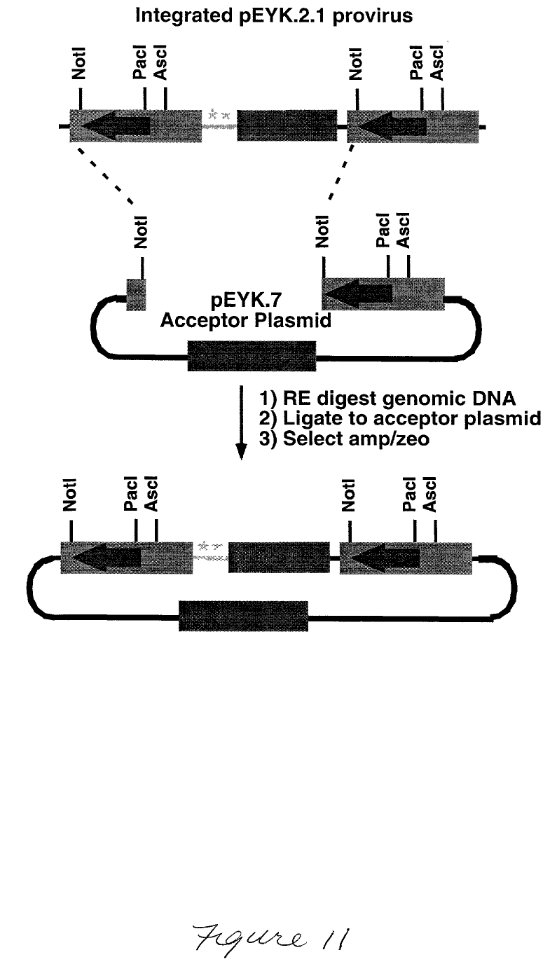

FIG. 12 depicts an iteration strategy for pEYK3.1 through the generation of sub-libraries. Cre-mediated excision or intramolecular ligation of restriction-enzyme digested genomic DNA is used to recover functional retroviruses and provide an enriched sub-library.

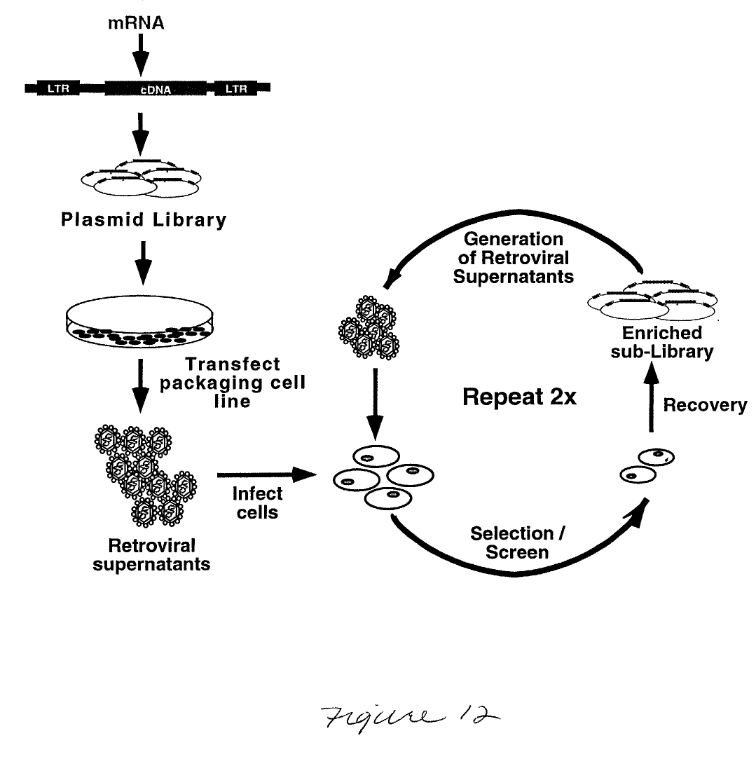

FIG. 13 shows reversion analysis of the pEYK3.1 vector subcloned with the BCR/ABL oncogene. FIG. 13A depicts an integrated B/A pEYK-3.1 provirus flanked by loxP sites. The B/A pEYK3.1 vector renders factor-dependent cell lines into factor-independent cell lines. FIG. 13B is a graph depicting reversion analysis with the B/A pEYK3.1 vector. The B/A pEYK3.1 vector was transformed in the presence of IL-3 with a polycistronic virus which expresses both Cre and the GFP-3M genes. Two days after Cre infection the population was divided in half, one half continued to receive IL-3 while the other half was deprived of IL-3. FACS analysis on the populations two days later demonstrated viability of the GFP-positive population grown in the absence of IL-3 decreased from 100% to 12%.

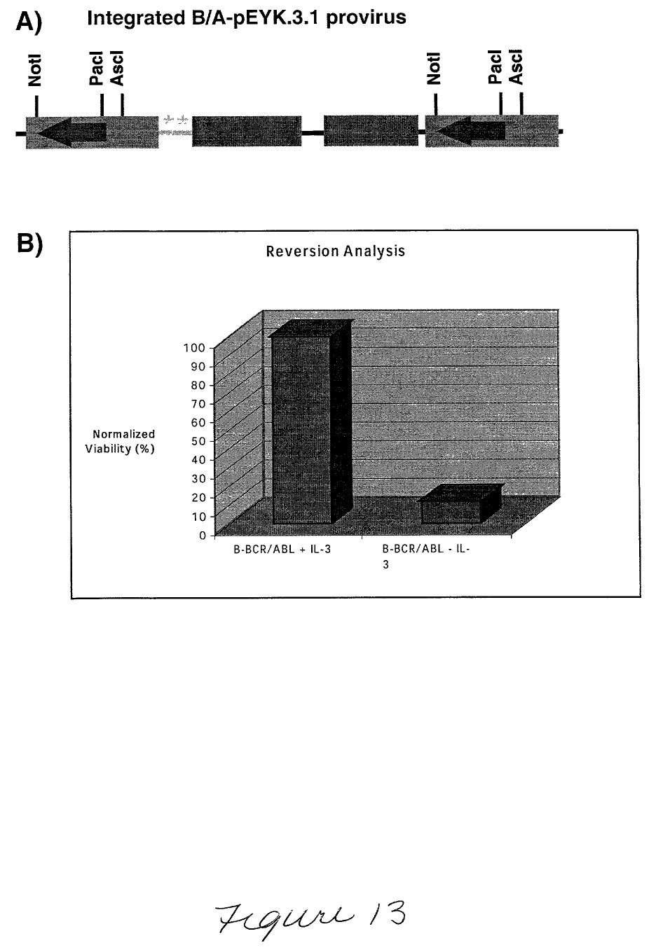

### DETAILED DESCRIPTION

The present invention is based, in part, on the development and characterization of expression vectors for phenotypic screens. These vectors provide the following benefits. They provide: (1) high viral titers to facilitate screening of a complete set of independent cDNAs, (2) high levels of gene expression, and (3) the ease of recovery of the desired cDNA. With these improvements, the pEYK retroviral vector systems offer significant advantages and improvements over current retroviral expression cloning systems.

Therefore, the invention includes viral vectors (e.g., retroviral vectors, e.g., replication deficient retroviral vectors), libraries comprising such vectors, retroviral particles produced by such vectors, retroviral packaging cell lines for production of these particles, integrated proviral sequences derived from the retroviral particles, circularized provirus sequences and mammalian cells upon which the provirus has been introduced.

The invention also includes methods of using such sequence, vectors, particles and cells. For example, the nucleic acid sequences described herein can be used to identify and isolate insert nucleic acids based upon their ability to complement a mammalian cellular phenotype, antisense based methods for identifying and isolating nucleic acids which inhibit or reduce function of a mammalian gene, and gene trapping methods to identify and isolate mammalian genes which are modulated in response to a specific stimuli.

Vectors

Described herein are vectors, e.g., retroviral vectors, useful in screening nucleic acid libraries. In one aspect, the vector can include a nucleic acid sequence. The nucleic acid includes from 5′ to 3′: a packaging sequence, a heterologous insert sequence or restriction sites for insertion of a heterologous sequence, and a 3′ LTR. The backbone of the vector can be, e.g., any vectors known in the art. For example, the vector is a retroviral vector. In one embodiment, the vector is a lentiviral vector. Lentiviral vectors can be used, for example, for proviral integration in post-mitotic cells. See, e.g., Frimpong et al. (2000) Gene Ther. 7:1562-1569; Naldini et al. (1996) Science 272:263-267; Naldini et al. (2000) Adv. Virus Res. 55:599-609.

Packaging Sequence

The packaging sequence has been altered to reduce the formation of fusion polypeptides encoded by the packaging sequence, or a portion thereof, and the heterologous insert sequence. A reduction in the formation of such fusion polypeptides can be obtained by altering at least one ATG codon of the packaging sequence. The packaging sequence can be altered such that the ATG initiation codon and at least one, two, three, or all of the internal ATG codon(s) has been altered. The ATG codon(s) can be altered such that one, two or all three nucleotide residues of the codon have been altered, e.g., substituted. Alteration of the ATG codon(s) can be obtained by methods known in the art such as site-directed mutagenesis of a known packaging sequence.

The nucleic acid sequence important for packaging can represent, for example, a gal/pol or an env gene sequence. In one embodiment, the packaging sequence is a gag packaging sequence, e.g., an amino-terminal portion of the gag sequence. For example, prior to alteration of at least one ATG codon, the packaging sequence can include all or a portion of the gag nucleotide sequence provided in SEQ ID NO:1. When the packaging sequence is a gag packaging sequence, one or more of the following nucleic acid residues can be altered such that an ATG codon is altered: one or more nucleic acid residues of the ATG initiation codon; one or more of the nucleic acid residues 1097-1099 of SEQ ID NO:1; one or more of the nucleic acid residues 1589-1591 of SEQ ID NO:1. For example, a gag packaging sequence used in the vector system is the altered gag packaging sequence of SEQ ID NO:2, or a portion thereof. The pEYK2.1 vector and the pEYK3.1 vector described herein include an altered packaging sequence as described above.

In one embodiment, use of a packaging sequence in which at least one, two or more, ATG codon(s) have been altered results in increased expression levels of the heterologous insert nucleic acid as compared to the same vector having a wild-type packaging sequence, e.g., the packaging sequence of SEQ ID NO:1. The expression levels can be increased by about 2, 3, 4, 5, 8, 10 or 20 fold as compared to vectors in which the packaging sequence has not been altered to reduce fusion polypeptide formation.

Long Terminal Repeat (LTR)

The vector further includes at least a 3′ LTR. The 3′LTR can be from, e.g., a retrovirus. For example, the 3′LTR can be from a Moloney murine leukemia virus (MoMLV), a mouse mammary tissue virus (MMLV), a murine stem cell virus (MSCV), a Rous Sarcoma virus (RSV), a feline leukemia virus (FLV), bovine leukemia virus, a spuma virus, a lentivirus (e.g., human immunodeficiency virus (HIV-1) and simian immunodeficiency virus (SIV)). In one embodiment, the 3′LTR includes one or more of a U3 region, a U5 region or a promoter containing portion thereof, an R region and a polyadenylation site.

In one embodiment, the 3′ LTR of the nucleic acid includes a proviral recovery sequence. The proviral recovery sequence allows for excision of retroviral provirus from the genome of a host cell, e.g., a mammalian host cell. The proviral recovery sequence can include at least one recombinase site and/or at least one, two, three, four, five or more, rare cutter restriction site(s). Examples of recombinase sites useful in the present invention include a loxP recombination site, mutants thereof, and an frt recombination site. The loxP recombination site is cleavable using a Cre recombinase enzyme. Contacting Cre recombinase to an intergrated provirus derived from the vectors described herein can result in excision of the proviral nucleic acid sequence. A description of the Cre/loxP recombinase system can be found, e.g., in Lasko et al. (1992) Prot. Natl Acad. Sci. USA 89:6232-6236. Alternatively, a mutant loxP recombination site can be used. For example, a loxP5 11 recombination site can be used which can only recombine with an identical mutant site. For a description of the loxP511 recombination site, see, e.g., Hoess et al. (1986) Nucleic Acid Res. 14:2287-2300. The frt recombination site is cleavable using a flp recombinase enzyme. A description of the frt/flp recombinase system can be found, for example, in O'Gorman et al. (1991) Science 251-1351-1355. Rare cutter restriction sites useful in the vector can include, for example, restriction sites which are at least 8 base pairs or larger. Examples of such restriction sites include, but are not limited to, a site fort Not1, SfiI, PacI and P1-SceI.

In one embodiment, the proviral recovery sequence is located within a portion of the 3′LTR which duplicates upon integration, e.g., duplicates such that the recovered provirus includes a 5′ LTR with a proviral recovery sequence, a 3′ LTR with a proviral recovery sequence, and a heterologous insert sequence between the two LTRs. For example, the proviral recovery sequence can be in the U3 region of the 3′ LTR. In another embodiment, the vector includes a 3′ LTR which includes a proviral recovery sequence and a 5′ LTR. When the vector includes only one proviral recovery sequence and acceptor plasmid, e.g., a pEYK7 vector as described herein, which also comprises a proviral recovery sequence. The acceptor plasmid can then be used for rescue of the vector having a proviral sequence in only one LTR.

As described above, the nucleic acid can further include a 5′ LTR which is 5′ from the heterologous insert sequence. The 5′ LTR can include one or more of: a U5 region or a promoter containing portion thereof, an R region, a U3 region and a primer binding site. The promoter can be, e.g., an internal LTR promoter or other inducible promoters. In one embodiment, the promoter is a cytomegalovirus (CMV) promoter. In one embodiment, the 5′ LTR includes, from 5′ to 3′, a U3 region, an R region and a promoter-containing portion of a U5 region. The 5′ LTR can further include a proviral recovery sequence, e.g., a 3′ LTR which includes a proviral sequence is duplicated upon intergration into the 5′ region of the nucleic acid such that the heterologous insert sequence is between the 5′ and 3′LTRs. The 5′ LTR can include any proviral recovery sequence described herein.

In another aspect, the nucleic acid sequence included in the vector can comprise a 5′LTR having a proviral recovery sequence, and a 3′ LTR which does not include a proviral recovery sequence. When the vector includes only one proviral recovery sequence, an acceptor plasmid, e.g., a pEYK7 vector as described herein, which also comprises a proviral recovery sequence can be used for rescue of the vector having a proviral sequence in only one LTR.

The 959 LTR

A series of retroviral vectors were created that allowed for more direct recovery of the provirus from the genomic DNA of mammalian cells. These vectors include a 3′ LTR having a proviral recovery sequence which is duplicated upon intergration to provide a 5′LTR having a proviral recovery sequence. An example of such an LTR is the 959 LTR described below.

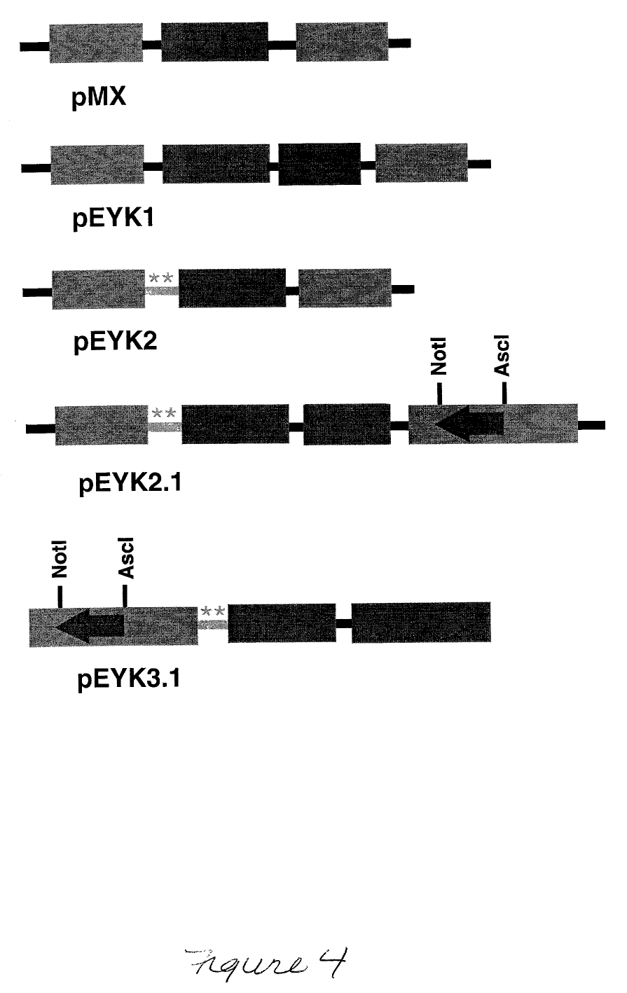

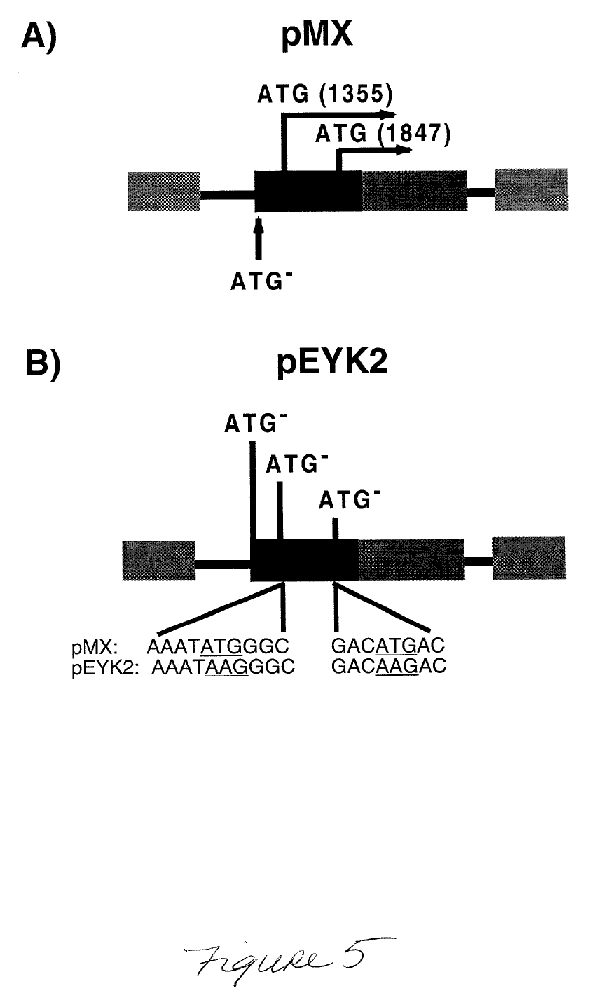

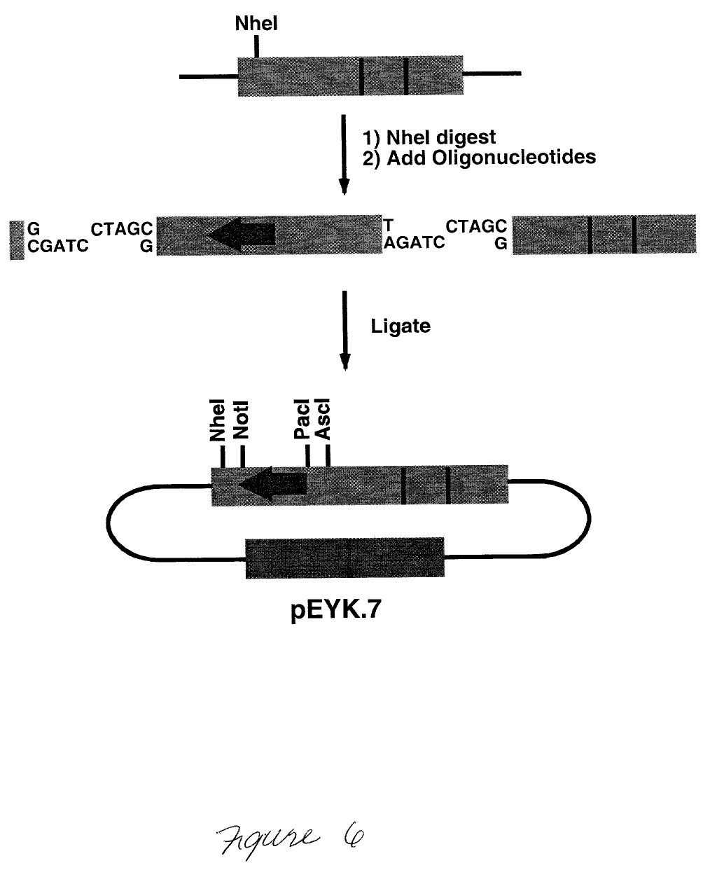

In order to recover and amplify the integrated provirus directly in bacterial cells, the retrovirus needed to contain a bacterial drug resistance sequence. Various bacterial drug resistance sequences are described herein. In addition, the isolation of the provirus from genomic DNA required specific sequences within the viruses that would allow the recovery of only the provirus and not any additional host DNA. In order to accomplish this method for recovery, the retroviral vectors were created to contain two identical rare-cutter restriction enzyme sites (for example, Not 1) and/or two loxP sites. Taking advantage of the life cycle of the retrovirus, the restriction enzyme sites and lox P sites were placed in the U3 region of the 3′ LTR (FIG. 5). Upon reverse transcription of the viral RNA, the 3′ U3 region of the long terminal repeat (LTR) can be copied over to the 5′ end to complete the LTR at the 5′ end (FIG. 6). The resulting retrovirus is thereby flanked by identical restriction enzyme sites and/or lox sites at the LTRs. Thus, the retrovirus has performed half the work by duplicating the restriction enzyme sites and lox P sites. In addition, placing these sites at the LTR allows one to isolate a fully functional provirus with the heterologous insert sequence. For example, Not I, loxP, and Asc I sites were placed in the Nhe I site of the U3 region (FIG. 4). The resulting vector pEYK7 was sequenced to check the integrity of the 959 LTR and the correct placement and orientation of the oligonucleotide insert.

Bacterial Origin of Replication

The nucleic acid can further include a bacterial replicon. The bacterial replicon includes a bacterial marker and an origin of replication (ori). The bacterial replicon facilitates the process of shuttling between mammalian and bacterial cells. In one embodiment, the nucleic acid includes only one origin of replication. It was found that amplification of a plasmid containing more than one origin of replication from the same complementation group was difficult. Examples of suitable bacterial origins of replication include pUC, colEI, pSCO101, p15, RK2 OriV, f1 phage Ori and the like. The origin of replication can be used in several different bacterial species, e.g., an ori which does not require a specific bacterial strain during replication. For example, the origin of replication, which can be used in several bacterial species, can be colEI. ColEI can be used, for example, in one or more of Bh5α, DH10B, JM109 and XL1blue.

The bacterial marker can be any of the selectable bacterial markers described herein. For example, the bacterial marker can be kanamycin/G418, zeocin, actinomycin, ampicillin, gentamycin, tetracycline, chloramphenicol and penicillin resistant markers.

The bacterial replicon can include a bacterial promoter, a bacterial marker and an origin of replication. In order to accommodate size limitation restrictions in retroviral packaging, the replicon can be less than 2 kb, 1.8 kb, 1.6 kb, 1.4 kb 1.2 kb, 1 kb in size. For example, the replicon can include a bacterial marker that is less than 600, 550, 500, or 450 kilobases in length. The bacterial replicon can include an EM7 promoter, a gene encoding bleomycin or mutants and fragments thereof, and a col1EI origin of replication.

Examples of Vectors

pEYK1

The pEYK1 vector includes a bacterial supF tRNA suppressor gene which can provide unique primer binding sites for PCR amplification and probes for Southern analysis and can allow direct recovery of the vector by selection in bacteria. The supF gene encodes a tRNA that allows translation read-through of amber stop codons. The supF gene was placed adjacent to the 3′ LTR in pMX. Upon transformation into the host MC1061/P3 bacteria cells that contain the P3 plasmid, which encodes the kanamycin resistance gene as well as amber mutants of tetracycline and ampicillin, the resulting vector pMX-supF vector grew slowly with an approximately 10-fold lower transformation efficiency of than the control pMX plasmid.

The low transformation efficiency was a hindrance to generating high complexity cDNA libraries in this vector. To document this, the pMX-subF vector was transformed into highly competent DH10B bacterial cells (>1010 colonies/ug). Under conditions where there was no selection for the supF gene, the bacterial colonies varied widely in size. Because the minute colonies represent clones that proliferate slowly, it is likely that expansion in liquid culture would lead to their under-representation, resulting in a skewed, biased library where certain cDNAs were either over- or under-represented. In addition, the difference in growth rates suggested that mutations had occurred within the plasmid to give a growth advantage. The most likely culprit was the supF gene, which, as a tRNA suppressor, would be toxic to the bacterial cells, allowing read-through of amber stop codons within the whole bacterial genome. The plasmids that grew better were sequenced; and one such clone, pEYK1, encoded a non-functional supF gene with six point mutations. The location of the six point mutations in the supF gene is provided in Table I. This mutant was designated subF. Although the shuttle capacity was compromised, the pEYK1 vector was useful because the subF sequence could be used as a probe for Southern analyses, as a target for PCR analyses to assay for titers, and finally as a unique sequence to design PCR primers flanking the cDNA insert.

TABLE I
Location of the Mutations found in SubF. Six point
mutations from the original supF gene were identified,
resulting in subF, a non-functional tRNA suppressor.

| SupF | Position | SubF |
|---|---|---|
| G | 34 | A |
| T | 39 | A |
| G | 41 | C |
| T | 43 | A |
| T | 48 | C |
| C | 94 | T |

pEYK2

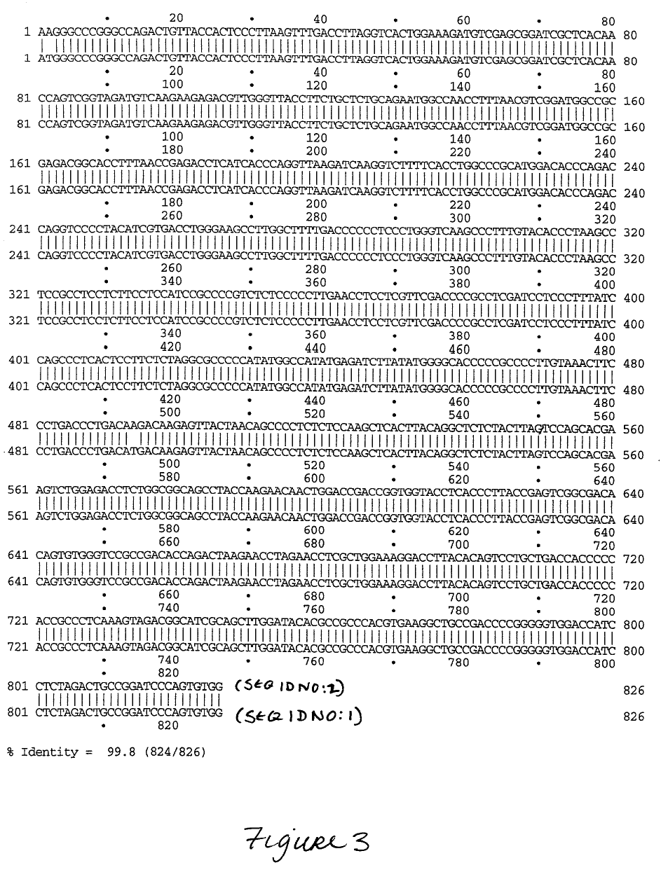

The pMX vector utilizes the extended packaging signal, which includes a 1 kb N-terminal portion of the gag gene. This inclusion of the 1 kb gag sequence has been shown to improve retroviral titers. Bender et al. (1 987) J. Virol. 61:1639-1646; Keller et al. (1 985) Nature 318:149-154. To avoid formation of gag region/polypeptide of interest fusion proteins, the ATG initiation codon and ATGs along the gag coding sequence which could potentially initiate translation were mutagenized. Open reading frame (ORF) analysis demonstrated two ATGs (1355 and 1847) that could initiate translation that would extend beyond the multiple cloning site (MCS) and into the cDNA coding sequence (FIG. 3A). To eliminate these two ATGs, two rounds of site-directed mutagenesis followed by careful sequencing and functional testing of the virus, resulted in the retroviral vector pEYK.2 (FIG. 3B).

pEYK2.1

As an alternative to PCR-based rescue, I created the pEYK2.1 retroviral vectors that would allow more direct recovery of the provirus from the genomic DNA of mammalian cells. Upon ligation to a bacterial replicon, this recovered provirus could then be amplified in bacterial cells (described in more detail below). In order to isolate and recover the integrated provirus, the cDNA insert needed to be linked to a bacterial drug resistance gene. Because of the size constraints in packaging a retrovirus, the marker needed to be of minimal size. Unfortunately, the small bacterial tRNA suppressor supF created problems when generating a library with a high number of independent colonies. The genes encoding for kanamycin resistance or ampicillin resistance were over 1 kb, restricting the size of the cDNA insert. On the other hand, the ble gene encoding for bleomycin/phleomycin resistance was ideal; the ble gene was about ˜420 bp, including the bacterial promoter (Drocourt et al., (1990) Nucleic Acid Res. 18:4009; Gatignol et al. (1988) FEBS Lett 230:171-175; Mulsant et al. (1988) Somat Cell Mol Genet. 14:243-252). When the ble gene including the EM7 bacterial promoter was cloned into pEYK2 vector, the bacterial colonies tolerated the ble gene in the retroviral vector; unlike the supF gene. No difference in size of the bacterial colonies that contained the pEYK2-ble construct was detected. To generate pEYK2.1, the 959 LTR replaced the 3′ LTR to generate a virus, when integrated in the genome, could be isolated by the flanking restriction enzyme sites (described in more detail below).

pEYK3.1

In a similar fashion to the episomal eukaryotic expression shuttle vectors, a completely self-contained bacterial replicon, containing both a marker and a bacterial origin of replication, could be placed into a retroviral vector. This self-contained bacterial replicon was hypothesized to facilitate the process of shuttling between mammalian and bacterial cells (described in more detail below). Because of the size limitation in retroviral packaging, the length of the replicon was a major concern. The EM7-ble-colE1 ori fusion was generated, creating a 1.1 kb fragment. A vector containing a single LTR provirus was generated that contained the EM7-ble-colE1 ori replicon (FIG. 4), resulting in the pEYK3 vector. Initial characterization of the pEYK3 vector, however, demonstrated low expression levels of the integrated provirus (described in more detail below). To improve the expression levels, the mutagenized gag sequence for pEYK2 replaced the corresponding sequence in pEYK3, yielding the pEYK3.1 vector that had significantly more expression than pEYK3 (described in more detail below).

Functional Characterization of the pEYK Vectors

Titers and Expression Levels

To test the function of each pEYK retroviral vector, a mutated green fluorescent protein was sub-cloned into each retroviral vector. GFP-3M contained the red-shifted, enhanced, and solubility mutations, making the GFP protein brighter and less toxic to the individual cells upon overexpression. With these GFP retroviral constructs, three independent transfections and infections of murine IL-3 dependent BaF/3 cells were performed as described in Kotani et al. (1994) Human Gene Ther. 5:19-28 to analyze retroviral titers and expression levels by measuring GFP fluorescence. In order to examine a population of infected cells with a single copy retrovirus, the infection percentage was targeted around 15-25%, which by Poisson statistics minimizes the number of cells that were infected by more than one retrovirus (Onishi et al. (1996) Exp. Hematol. 24:324-329). Table 2 lists the average titers with standard deviations for each pEYK vector. To examine expression levels, the infected population underwent FACS (fluorescent activated cell sorting) analyses; the geometric mean of fluorescence intensity of GFP was divided by autofluorescence in order to obtain the fold increase in expression above background.

TABLE 2
Comparative analyses of retroviral titers and expression
levels. Retroviral supernatants (50 uL) were used to infect
1 × 10⁶ BaF/3 cells. Independent transfections and
infections were repeated three times for each retroviral
construct. The transfection efficiencies of the 293T packaging
cells were roughly identical for all retroviral constructs
(65%-70%). The numbers listed in the table represent the
average and standard deviations of the three experiments.

| | | Fold Expression Above |
|---|---|---|
| Vector | Viral Titer (×10⁶) | Background |
| pMX | 6.6 ± 0.5 | 315 ± 13 |
| pEYK1 | 6.2 ± 0.3 | 237 ± 5 |
| pEYK2 | 6.5 ± 0.3 | 382 ± 26 |
| pEYK2.1 | 6.7 ± 0.2 | 175 ± 12 |
| pEYK3 | 1.0 ± 0.07 | 33 ± 1 |
| pEYK3.1 | 1.0 ± 0.05 | 121 ± 8 |

Comparison of Expression Levels of pEYK2 and pMX

The initial rationale behind pEYK2 was the elimination of two internal ATGs within the 1 kb gag coding sequence in pMX to prevent gag-cDNA protein fusions. After eliminating the two ATGs, the pEYK2 construct has a significant increase in expression levels when compared to pMX (382 fold above background vs. 315 fold above background). Anticipating that each modification added to the virus may have detrimental effects on the expression levels and titers, each subsequent pEYK vector was derived using this mutagenized gag region from pEYK2, which augmented expression levels.

LTR vs. modified LTR (959 LTR)

In order to generate alternative methods of recovery, the U3 region of the 3′ LTR was modified to contain three restriction enzyme sites and a loxP site, resulting in the 959 LTR. The functional integrity of the 959 LTR was tested as follows. In FIG. 5, retroviral constructs (pEYK2.2 and pEYK2.3) containing identical elements with the only exception being the absence (pEYK2.2) or the presence (pEYK2.3) of the 959 LTR were analyzed for both expression levels and retroviral titers. In comparing pEYK2.2 and pEYK2.3, there were no significant differences in either the expression levels or the titers, demonstrating the 959 LTR had no detrimental effect on expression level and titers of recombinant retroviral vectors that utilize this modified LTR.

pEYK3 vs. pEYK3.1

The pEYK3 and pEYK3. 1 vectors are a radical change from the traditional retroviral plasmid vectors—not only was there a bacterial replicon (EM7-ble-colE 1 fusion) placed within the virus, but also the vectors contained only a single LTR (959 LTR). The presence of the bacterial replicon dramatically decreased the expression levels; in pEYK3, the fold increase in fluorescence was only 33 fold above background fluorescence (FIG. 6). In addition, the titers were reduced to 1×10⁶ IFU/mL, although these were still adequate for expression cloning strategies. To improve the expression levels, the mutagenized gag sequence from the pEYK2 vector was utilized, generating the pEYK3.1. Although almost half as much as the other pEYK vectors, the expression levels of pEYK3.1 were significantly improved—over 4 times the fluorescent levels of the parental pEYK3 vector.

Recovery

In any expression cloning strategy, one important step is the ability to recover the cDNA insert that is responsible for the screened phenotype. Each of the vectors described herein utilizes a unique strategy to isolate the cDNA insert through PCR-based rescue of the cDNA insert, restriction enzyme excision of the provirus from genomic DNA, or finally cre-mediated recovery of the provirus.

PCR Rescue

The initial efforts to use the retroviral expression cloning system pMX described by Onishi et al. (1996) Mol. Cell Biol. 18:3871-3879 were plagued by the inability to PCR amplify the cDNA insert using the primer pairs published in the paper without non-specific amplification. The pMX vector contained only 35 base pairs between the cDNA and the 3′LTR; with such a short stretch of sequence, the design and optimization of PCR primers flanking the cDNA insert is difficult. In addition to primer design, the length of the cDNA insert and the GC content of the cDNA insert are important factors in determining conditions for the PCR reaction. Because both of these factors are unknown in rescuing an unidentified cDNA insert, the PCR amplification is essentially a “blinded” process. Because of these several unknown variables, designing the primers became important in order to eliminate amplification of non-specific sequences. In addition, the mouse and human genome contain retroviral-like elements, such as endogenous retroviruses and LINE and SINE elements that serve as non-specific templates in the PCR amplification process. In the pEYK1 vector system, the addition of the subF sequence allowed for the development of primer pairs for the PCR reactions to be more efficient and more specific. For primer design, fifteen primer pairs were chosen using the Primer 3 program (http://www-genome.wi.mit.edu/cgi-bin/primer/primer3.cgi) with each primer being compared against the murine repetitive sequence database from the Whitehead Genome Center to ensure minimization of non-specific primer binding with the mouse genome (Table 3). The best pair (Primer Pair 5) was 1759 and 3289. These primers were used to amplify the cDNA inserts from a primary screen attempting to identify cDNAs that would complement cytokine signaling. In comparison to the PCR reactions from pMX-library infected cells, the PCR reactions from pEYK1-library infected cells demonstrate the high-specificity of the PCR amplification with distinct bands and minimal non-specific background products.

TABLE 3

Primer Pair 1

| | | |
|---|---|---|
| 1759 | AAAGGACCTTACACAGTCCTGCTGA | (SEQ ID NO:3) |
| 3291 | CACCACAGGTAATGCTTTTACTGGC | (SEQ ID NO:4) |

Primer Pair 2

| | | |
|---|---|---|
| 1763 | GACCTTACACAGTCCTGCTGACCAC | (SEQ ID NO:5) |
| 3291 | CACCACAGGTAATGCTTTTACTGGC | (SEQ ID NO:6) |

Primer Pair 3

| | | |
|---|---|---|
| 1738 | AAGAACCTAGAACCTCGCTGGAAAG | (SEQ ID NO:7) |
| 3291 | CACCACAGGTAATGCTTTTACTGGC | (SEQ ID NO:8) |

Primer Pair 4

| | | |
|---|---|---|
| 1759 | AAAGGACCTTACACAGTCCTGCTGA | (SEQ ID NO:9) |
| 3344 | GAAGTCGATGACGGCAGATTTAGAG | (SEQ ID NO:10) |

Primer Pair 5

| | | |
|---|---|---|
| 1759 | AAAGGACCTTACACAGTCCTGCTGA | (SEQ ID NO:11) |
| 3289 | CCACAGGTAATGCTTTTACTGGCCT | (SEQ ID NO:12) |

Primer Pair 6

| | | |
|---|---|---|
| 1760 | AAGGACCTTACACAGTCCTGCTGAC | (SEQ ID NO:13) |
| 3291 | CACCACAGGTAATGCTTTTACTGGC | (SEQ ID NO:14) |

Primer Pair 7

| | | |
|---|---|---|
| 1763 | GACCTTACACAGTCCTGCTGACCAC | (SEQ ID NO:15) |
| 3344 | GAAGTCGATGACGGCAGATTTAGAG | (SEQ ID NO:16) |

Primer Pair 8

| | | |
|---|---|---|
| 1763 | GACCTTACACAGTCCTGCTGACCAC | (SEQ ID NO:17) |
| 3289 | CCACAGGTAATGCTTTTACTGGCCT | (SEQ ID NO:18) |

Primer Pair 9

| | | |
|---|---|---|
| 1724 | GCCGACACCAGACTAAGAACCTAGA | (SEQ ID NO:19) |
| 3291 | CACCACAGGTAATGCTTTTACTGGC | (SEQ ID NO:20) |

Primer Pair 10

| | | |
|---|---|---|
| 1759 | AAAGGACCTTACACAGTCCTGCTGA | (SEQ ID NO:21) |
| 3296 | AACCCCACCACAGGTAATGCTTTTA | (SEQ ID NO:22) |

Primer Pair 11

| | | |
|---|---|---|
| 1763 | GACCTTACACAGTCCTGCTGACCAC | (SEQ ID NO:23) |
| 3296 | AACCCCACCACAGGTAATGCTTTTA | (SEQ ID NO:24) |

Primer Pair 12

| | | |
|---|---|---|
| 1759 | AAAGGACCTTACACAGTCCTGCTGA | (SEQ ID NO:25) |
| 3287 | ACAGGTAATGCTTTTACTGGCCTGC | (SEQ ID NO:26) |

Primer Pair 13

| | | |
|---|---|---|
| 1759 | AAAGGACCTTACACAGTCCTGCTGA | (SEQ ID NO:27) |
| 3222 | GCCGCTGTAAAGTGTTACGTTGAGA | (SEQ ID NO:28) |

Primer Pair 14

| | | |
|---|---|---|
| 1763 | GACCTTACACAGTCCTGCTGACCAC | (SEQ ID NO:29) |
| 3287 | ACAGGTAATGCTTTTACTGGCCTGC | (SEQ ID NO:30) |

Primer Pair 15

| 1763 | GACCTTACACAGTCCTGCTGACCAC | (SEQ ID NO:31) |
|---|---|---|
| 3222 | GCCGCTGTAAAGTGTTACGTTGAGA | (SEQ ID NO:32) |
| Table 3: PCR primer pairs for pEYK1. For primer design, fifteen primer pairs were chosen using the Primer 3 program (http://www-genome.wi.mit.edu/cgi-bin/primer/primer3.cgi) with each primer being compared against the murine repetitive sequence database from the Whitehead Genome Center to ensure minimization of non-specific primer binding with the mouse genome. Primer pair 5 was the best primer pair during test PCR amplifications of known cDNA inserts incorporated into the murine genome of | | |
| # BaF/3 cells. | | |

To further characterize the ability to recover a specific cDNA insert from a single-copy provirus, genomic DNA was isolated from a sorted population of BaF/3 cells that were infected with low titer pEYK1-GFP virus. Using conditions as identical for the rescue of unknown cDNAs, PCR amplifications were performed on serial dilutions of the genomic DNA with single-copy retrovirus starting from 100 ng of template. These serial dilutions of genomic DNA template revealed that the limit of detection with 35 rounds of amplification was 2 ng of genomic DNA. More cycles could be have been used, but increasing the number of cycles also resulted in the amplification of non-specific background products. To reproduce more faithfully the conditions in an actual screen, serial dilutions of genomic DNA with single-copy retrovirus were performed in the presence of uninfected BaF/3 genomic DNA. In this experiment, the limit of detection was still 2 ng of genomic DNA with single-copy retrovirus. With primer design optimization, the pEYK1 vector system allowed for specific amplification of cDNA inserts, resulting in significant improvements in the PCR-based recovery.

Restriction Enzyme-Mediated Rescue

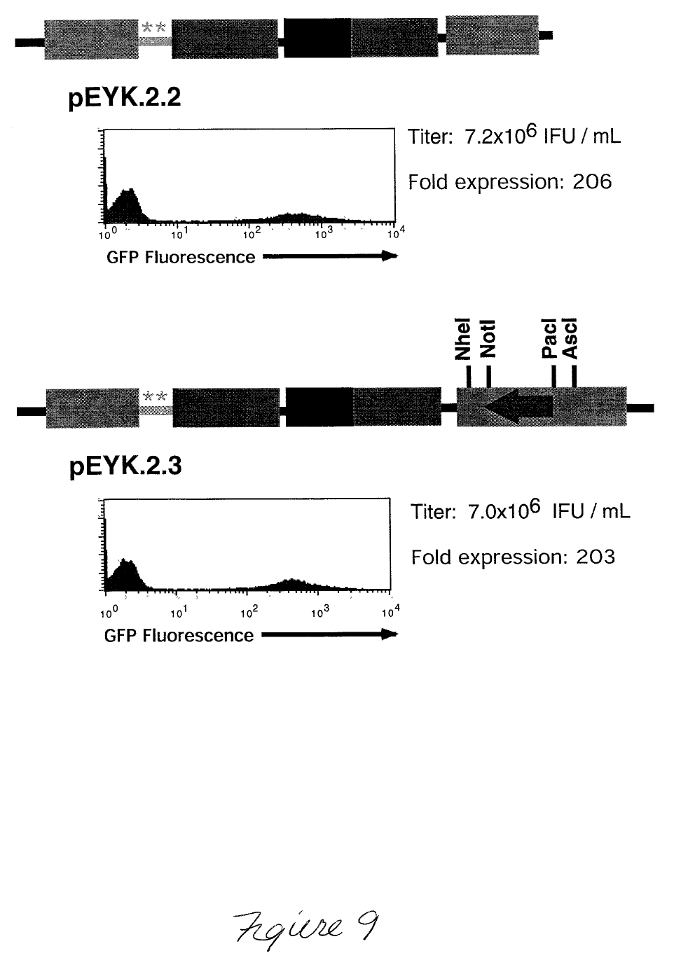

In addition to various problems with PCR amplification, cumbersome steps are required to subclone the cDNA insert into the retroviral vector in order to regenerate the provirus. Therefore, rather than just rescuing the cDNA insert, the nucleic acids of the invention allow for the rescue a fully functional provirus that could be repackaged and efficiently reintroduced back in to mammalian cells. Such a process eliminates the time-consuming process of subcloning the PCR product. The recovery strategy for pEYK2.1 and pEYK3.1 allow for both the rescue of the cDNA insert and the regeneration of the provirus. For the pEYK2.1 vector system, recovery of the integrated provirus from genomic DNA entails restriction enzyme digestion with one of the flanking restriction enzyme sites in the 959 LTR (either Not I or Asc I). Next, the digested genomic DNA is ligated to an acceptor plasmid (pEYK7), resulting in a reconstituted virus that can be selected and amplified in the presence of both ampicillin and zeocin (FIG. 9). The double drug selection eliminates the product of the self-ligation of pEYK7, which would confer only ampicillin resistance. The observed rescue efficiency ranged from 50-200 colonies per 1 μg of genomic DNA containing single-copy retrovirus (Table 4). Upon restriction enzyme analyses of the recovered virus, roughly 50% of the products were the legitimate virus with the accurate restriction map. The other products were a result of an unknown recombination event. Further analyses are in progress to determine the nature of this presumably recombinant vector. The regenerated proviruses were fully functional. Miniprep DNA from the recovered virus was co-transfected with the pCL-Eco packaging construct in order to generate retroviral supernatants. The isolated supernatants were then used to infect BaF/3 cells, resulting in a population becoming GFP positive. These results demonstrate that the recovered virus is fully functional with titers and expression levels equivalent to the parental vector.

For the pEYK3.1 vector system, recovery of the integrated single-copy provirus from genomic DNA entails restriction enzyme digestion with either Not I or Asc I—sites that were engineered in the LTR. Next, the digested genomic DNA is diluted and ligated in conditions favoring self or intramolecular ligations, rather than intermolecular ligation products. The resulting reconstituted virus that can be selected and amplified in the presence of zeocin. The efficiency was consistent and extremely high (Table 4). The recoverability was close to that achieved by PCR amplification in the pEYK. 1 vector system and may be even slightly higher with the ability to obtain one bacterial colony with less than 2 ng of genomic DNA. Restriction enzyme analyses of the rescued viruses demonstrated a high recovery (39/40) of fully functional viruses. The minipreps of the recovered pEYK3.1 virus generated functional retrovirions that were able to infect BaF/3 cells with the same titers and expression levels as the parental vector.

Cre-Mediated Rescue

An alternative strategy to rescue the integrated pEYK3.l provirus from genomic DNA is the utilization of the cre recombinase to excise the DNA as a circular form that can be immediately transformed into bacteria for amplification (Ringrose et al. (1998) J. Mol. Biol. 284:363-384). With optimized conditions, greater than 97% of the recovered products had generated a fully functional retrovirus upon restriction map analyses and functional testing of the supernatants (data not shown). The cre-mediated recovery, however, was less efficient than the self-ligation of digested genomic DNA (Table 4).

Cloning Efficiency: 1 in 10⁶ Recovery for pEYK1 and pEYK3.1

Mock screens were established in order to test the functional cloning efficiencies of the retroviral vectors pEYK1 and pEYK3.1. The murine c-mpl gene (the cytokine receptor for TPO-thrombopoietin) and GFP-3M were subcloned into the pEYK1 and pEYK3.1 retroviral vectors. When introduced into the murine interleukin-3 (IL-3)-dependent BaF/3 cells, the c-mpl gene confers TPO-dependent cell growth in the absence of any IL-3. The pEYK1-c-mpl vector and the pEYK3.1-c-mpl vector were serially diluted into the background of pEYK1-GFP and pEYK3.1-GFP vectors, respectively. Retroviral supernatants were generated, and BaF/3 cells were infected. Then, the infected BaF/3 cells were selected in the presence of TPO and in the absence of IL-3. Both vector systems were able to isolate the c-mpl cDNA even when the c-mpl retroviral vectors were represented in a frequency of 1 in 106 (data not shown). Previous retroviral expression cloning systems were ten-fold less sensitive (Kitamura et al. (1995) Proc. Natl Acad Sci USA 92:9146-9150; Rayner and Gonda (1994) Mol. Cell Biol. 14:880-887). With these higher cloning efficiencies, both the pEYK1 and the pEYK3.1 vectors systems have higher sensitivity in functionally detecting genes that are represented in low abundance in cDNA libraries. Therefore, both pEYKI and pEYK3.1 provide significant improvements over current systems.

Iteration Strategies for Phenotypic Screens

pEYK1: Helper Virus Rescue

In any expression cloning strategy, the ability to iterate/repeat the screen is important to enrich for candidates that are true positives. For the pEYK1 vector system, the inability to recover the whole provirus made the whole iterative process cumbersome. Two strategies exist to regenerate the retrovirions for an iterative screen. First, the cDNA insert can be subcloned into a cloning vector using, e.g., a TOPO TA cloning kit (Invitrogen) and then subcloned back into the pEYK1 vector. Alternatively, the selected/screened population can be superinfected with replication competent wild-type Moloney virus in order to mobilize the integrated proviruses. The selected/screened cells now become the packaging the cell line, liberating both wild-type virus and the integrated proviruses. Then, the screen/selection can be repeated by infection of fresh, uninfected cells. The ability to mobilize the integrated virus depends on the cell lines (Miller et al. (1985) Mol. Cell Biol. 5:431-437). Various cell lines were generated with pEYK1-GFP retrovirus. A pure GFP-positive population was derived using FACS sorting. Each GFP-positive cell line was infected with wild-type Moloney virus that was generated from the pZAP construct (Shoemaker et al. (1981) J. Virol. 40:164-172). Supernatants were isolated three days later and infected on fresh target cells. The titers of the mobilized pEYK1-GPF provirus ranged from 10² to 10³ IFU/mL. The low titers of the mobilized provirus may not provide enough virions to infect a high number of cells. With such a low number of infected cells, the length of subsequent screens/selections may not be significantly shorter.

pEYK3.1: Sub-Library Generation

The ease of recovering fully functional provirus is an advantage of the pEYK3.1 system over existing retroviral expression cloning systems. To iterate the screen for pEYK3.1 retroviral vector system, cre-mediated excision from genomic DNA or self-ligation of restriction-enzyme digested genomic DNA generates fully functional retroviruses that can immediately be used to generate retroviral supernatants. The efficiency in generating enriched sub-libraries provides the pEYK3.1 vector system with a significant advantage over traditional methods of PCR recovery and subsequent subcloning steps to re-create the provirus. In addition, the recovered virus has titers identical to the parental vector, thereby allowing infection of high numbers of cells. With approximately a thousand-fold higher titers than ones obtained through mobilization with wild-type Moloney virus, the pEYK3.1 system substantially enriches for the number of infected cells, thereby shortening the length of subsequent screens/selections.

Reversion Analysis

Besides the ease of recovering the integrated provirus from the genomic DNA of infected cells, the pEYK3.1 vector system also offers the capability to perform reversion analyses to confirm the phenotypes of the cDNAs. Because the integrated provirus is flanked by loxP sites, the cre gene can be introduced retrovirally into the cells containing the provirus. The cre enzyme can now mediate in vivo excision of the provirus. The cre-infected cells no longer express the cDNA and subsequently revert back to the parental phenotype. To demonstrate the reversion capability of pEYK3.1, the BCR/ABL oncogene was subcloned into the pEYK3.1 vector, generating the pEYK3.1-B/A vector. Upon infection of the pEYK3.1-B/A vector, the IL-3-dependent BaF/3 cells proliferate and survive in the absence of IL-3 (Daley and Baltimore (1988) Prot Natl Acad Sci USA 85:9312-9316). In the presence of IL-3, this BCR/ABL-transformed population is then infected with a bicistronic virus expressing both the cre and GFP-3M genes. Two days after cre infection the population was divided in half: one-half of the population continued to receive IL-3; while the other half was deprived of IL-3. FACS analyses on the populations two days later demonstrated that the GFP-positive population decreased from 100% viability (normalized) to 12% viability (normalized) when the population was deprived of IL-3. Normalized values were used to eliminate the decreased viability due to the toxic overexpression of the cre enzyme. The decrease in normalized viability demonstrates that the in vivo excision of the pEYK3.1-B/A provirus is reverting the cells back to cytokine dependency. Without the presence of IL-3, the cells were unable to survive and were eliminated through apoptosis.

Library Generation

The nucleic acid vectors described herein can be used to screen and identify nucleic acids from a library. Thus, in one aspect, the invention features a method of generating a library. The method includes: (1) providing an insert nucleic acid library (e.g., a cDNA library); (2) inserting at least a portion (i.e., a sub-library) of the nucleic acids from the library into a nucleic acid vector described herein. The method can also include introducing the sub-library into mammalian cells, e.g., cells of a packaging cell line. The cell can be adapted to expresss a retroviral envelope (env) protein and/or a retroviral reverse transcriptase (pol). Preferably, the cell is unable to produce a wildtype retrovirus, e.g., the cell lacks a gene encoding a gag polypeptide. The method can also include harvesting retroviral particles containing a nucleic acid as described herein.

In one embodiment, the method of generating the library further includes separating the insert nucleic acids into at least two sub-libraries prior to insertion of the nucleic acids into a vector, and then inserting each of the sub-libraries into a nucleic acid vector described herein. The nucleic acid library can be separated into sub-libraries based upon the size of the insert nucleic acid. By separating based upon size, preferential amplification of smaller nucleic acids can be reduced. For example, the nucleic acid library can be separated into sub-libraries having insert nucleic acids of about 1 kb or less, and those with insert nucleic acids greater than about 1 kb. In another embodiment, the nucleic acid library is separated into at least three sub-libraries: insert nucleic acids of about 500 basepairs or less, insert nucleic acids of about 1 to 3 kb, and insert nucleic acids greater than about 3 kb. The nucleic acid library can be subjected to size fractionalization, e.g., using SDS-PAGE, and separated based upon size into at least two, three, four sub-libraries.

The library generated can be: a normalized or non-normalized library for sense or antisense expression; a library selected against a specific chromosome or region of a chromosome (e.g., YACs); a library generated from any tissue source, e.g., from healthy or diseased tissue.

The library can be generated by known methods. For example, to convert mRNA to cDNA, Superscript Choice System cDNA synthesis kits (Life Technologies) were utilized with modifications. For a typical cDNA synthesis, the cDNAs ranged in length from 100 bp to 12 kb. The cDNAs were ligated with BstX1 adaptors.

In order to obtain a non-biased bacterial amplification of the libraries, the cDNA products were size-fractionated in order to prevent preferential amplification of smaller cDNAs by the bacterial host. Various methods of size fraction were tested, and the method with the best recovery was the utilization of low-melt agarose gel with subsequent digestion with agarase enzyme. The standard size fractionation with Sephacryl columns provided poor yields of recovery (data not shown).

The cDNA syntheses were size-fractionated into 3 major groups: about 500 bp to 1 kb, about 1 kb to 3 kb, and >3 kb. Two of the fractionations (1 kb-3 kb and >3 kb) were subsequently ligated into the non-palindromic BstX1 sites of pEYK1, pEYK2.1, or pEYK3.1. in a non-directional fashion. The 500-bp to 1 kb fraction was not used because a majority of this fraction contained incomplete cDNA fragments. After the ligation, the two size-fractions were separately amplified in bacteria either by limited growth in liquid cultures or by expanding the library on multiple large plates. For each sub-library the total number of independent cDNAs was approximately 1×10⁶.

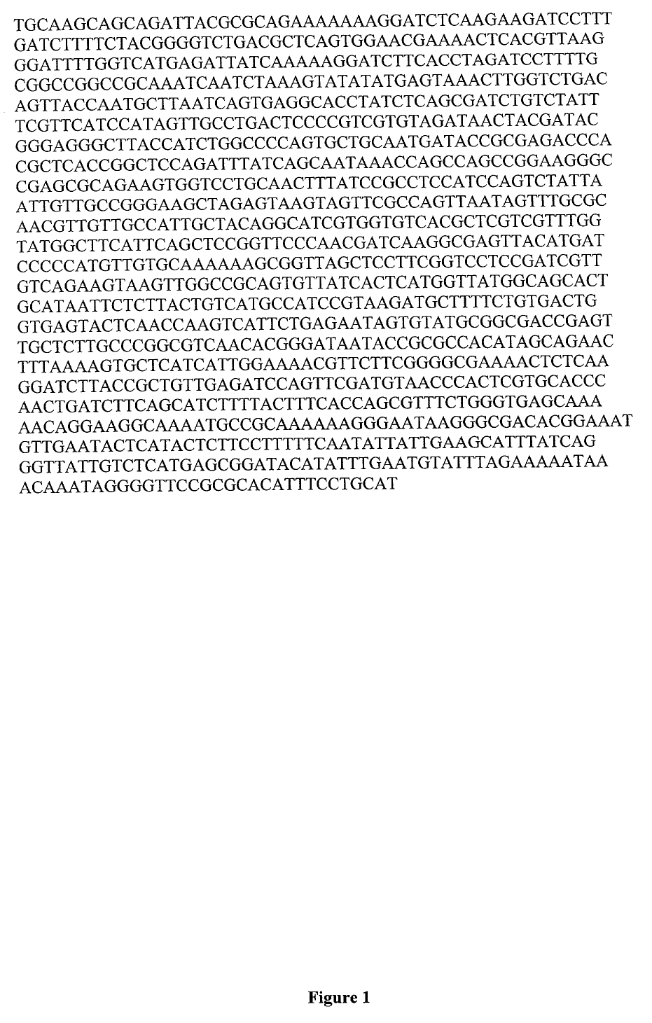

In the amplification using large plates, each sub-library was further divided into 4-5 pools. Each pool was characterized for the average size of cDNA inserts and for size range. The DNA was digested with NheI, which cuts at both LTRs in the pEYK1 and pEYK2.1 vector systems, liberating the backbone of the vector (2.6 kb) and the cDNA, which contains a 2 kb portion of the retrovirus. Typically, the 1-3 kb sub-library generated an average insert size of 1.5 kb with a range from 500 bp to 3 kb (FIG. 1 2B). For the >3 kb sub-library, the average size of the cDNA insert was 3 kb with a range from 1 kb to 8 kb. Several libraries have been generated in the various retroviral vectors pEYK1, pEYK2.1, and pEYK3.1 vector systems (Table 5).

TABLE 5
Retroviral cDNA libraries. Various sources of cDNAs
have been placed into the pEYK vector systems. PV and
ET samples are derived from the peripheral blood of patients
with polycythemia vera (PV) and essential thrombocythemia
(ET), respectively. ET and PV are myeloproliferative disorders.
For each of the libraries, the sub-libraries (3+ and 1-3 kb)
contain a range from 8 × 10⁵ to 1.2 × 10⁶ independent cDNAs.

| cDNA source | pEYK.1 | pEYK.2.1 | pEYK.3.1 |
|---|---|---|---|
| K562 (human erythroleukemia cell line) | x | | x |
| JEG3 (human choriocarcinoma cell line) | x | | x |
| U20S (human osteosarcoma cell line) | x | | x |
| VA13 (human lung fibroblast cell line) | x | | x |
| LMJ216 (human fibroblast cell line) | x | | x |
| BJ (human foreskin fibroblast) | | | x |
| Jurkat (human T-cell line) | | | x |
| MCF-7 (human breast cancer cell line) | | | x |
| D14 murine fetal liver | x | | |
| D14 Whole Embryo | | | x |
| RS (PV Patient) | x | | |
| DA (PV Patient) | x | | |
| SB (PV Patient) | x | x | |
| DM (PV Patient) | | | x |
| 4706 (PV Patient) | x | | |
| TC (ET Patient) | x | | |

The compositions of the present invention further include libraries comprising a multiplicity of the retroviral vectors of the invention, said retroviral vectors further containing cDNA or gDNA sequences. A number of libraries may be used in accordance with the present invention, including but not limited to, normalized and non-normalized libraries for sense and antisense expression; libraries selected against specific chromosomes or regions of chromosomes (e.g., as comprised in YACs or BACs), which would be possible by the inclusion of the f1 origin; and libraries derived from any tissue source.

Packaging Cell Lines

Various known retroviral packaging cell lines can be used to package retroviral-derived nucleic acids described herein into replication-deficient retroviral particles capable of infecting appropriate mammalian cells. Such packaging cell lines are described, for example, in Danos et al. (1988) Proc. Natl Acad. Sci USA 85:6460-6464; Markowitz et al. (1988) Virology 167:400-406; Chong et al. (1996) Gene Ther. 3:624-629; Cossette et al. (1995) J. Virol. 69:7430-7436; Rigg et al. (1996) Virology 218:290-295; and, U.S. Pat. No. 6,025,192, the contents of which are incorporated herein by reference. The retroviral packaging functions can include gag/pol and env packaging functions. Gag and pol provide viral structural components and env functions to target virus to its receptor. Env function can include an envelope protein from any amphotropic, ecotrophic or xenotropic retrovirus, including but not limited to MuLV (such as, for example, an MuLV 4070A) or MoMuLV. Env can further include a coat protein from another virus (e.g., env can comprise a VSV G protein) or any molecule that targets a specific cell surface receptor.

Screening Methods Using the Nucleic Acids Sequences, Vectors and Particles

The vectors described herein can be used in various screening methods to identify and isolate insert nucleic acids having particular functions. For example, the vectors can be used to identify (and isolate) nucleic acids based upon their ability to complement a mammalian cell phenotype, using antisense methods to identify (and isolate) nucleic acids which inhibit or reduce the function of a mammalian gene, and by methods to identify (and isolate) mammalian genes which are modulated, e.g., abrogated or enhanced, in response to a specific stimuli.

The compositions also include retroviral vectors, e.g., replication deficient retroviral vectors, such as complement screening vectors, antisense-genetic suppressor element (GSE) vectors, vectors displaying random peptide sequence, libraries which include such vectors, retroviral particles produced by such vectors and packaging cell lines.

Complementation Screening Methods

Mammalian cell complementation screening methods can include, for example, a method for identification of a nucleic acid sequence whose expression complements a cellular phenotype. Such methods can include: (a) infecting a mammalian cell exhibiting the cellular phenotype with a retrovirus particle derived from an insert nucleic acid-containing retroviral vector described herein, wherein, upon infection an integrated retroviral provirus is produced and the insert nucleic acid is expressed; and (b) analyzing the cell for the phenotype, so that suppression of the phenotype identifies an insert nucleic acid sequence which complements the cellular phenotype. The term “suppression”, as used herein, refers to a phenotype which is less pronounced in the presence in the cell expressing the insert nucleic acid as compared to the phenotype exhibited by the cell in the absence of such expression. The suppression may be quantitative, e.g., in changing the rate of cell growth or level of expression of a marker gene or protein, or qualitative, e.g., a change in cell shape or migration, and will be apparent to those of skill in the art familiar with the specific phenotype of interest.

In another embodiment, a nucleic acid which complements a phenotype of a mammalian gene can be identified, e.g., screened for, using knock out cells. These screens entail complementing a knock out phenotype with a candidate insert nucleic acid other than the targeted knock out gene. Examples of known knock out cells such as acetylcholinesterase knock out cells, adenylate cyclase 1 knock out cells, adenosine receptor knock out cells, to name a few, are described, e.g., in Bolivar et al. (2000) Mamm. Genome 11:260-274, Muller et al. (1999) Mech. Dev. 82:3-21 and at http//www.wadsworth.org. Examples of other knock out genes which have been used in phenotypic screens include genes involved in cell growth or senescence. Bems et al. (2000) Oncogene 19:3330-3334 have described, e.g., screens to rescue biological defects of c-myc knock out fibroblasts from the slow-growth phenotype. In addition, pEYK vectors described herein have been used to screen for insert nucleic acids which rescue bmi-1-null fibroblasts from premature senescence. Other rescue screens include, but are not limited to, identifying insert nucleic acids which rescue ras-induced premature senescence, arf-induced arrest, immortilization, radiation resistance, prostate tumorigenecity, angiogenesis (e.g., recruitment of endothelial cells), invasiveness, anchorage independence, drug-resistance, inhibited differentiation, TGF-β resistance, and apoptosis.

The present invention also includes methods for the isolation of nucleic acid molecules identified via the complementation screening methods of the invention. Such methods can utilize PCR-mediated rescue or the proviral recovery sequences in the restriction enzyme mediated or Cre-mediated methods as described herein.

Lethal Selection

One method of complement screening which can be used is a lethal selection method which relies on the candidate insert nucleic acid conferring a survival or proliferative advantage over a negative population. See, e.g., Stark et al. (1999) Human Mol. Genet. 8:1925-1938. Lethal selections can significantly eliminate background noise in the screening procedure to help distinguish true positives from false positives. For example, apoptosis-inducing agents (e.g., radiation, cytotoxic drugs, TGF-β, etc.) will cull the population that does not express the appropriate candidate to allow the cells to by-pass crisis. Lethal selections can include selection screens which allow a cell to bypass senescence and crisis, see, e.g., Hahn et al. (1999) Nature 400:464-468; Montalto et al. (1999) J. Cell Physiol. 180:46-52; Bryan et al. (1997) Nat. Med. 3:1271-1274; and Reddel et al. (1997) Biochemistry 62:1254-1262, or allow survival in anchorage independent conditions, see, e.g., Schwartz et al. (1997) J. Cell Biol. 139:575-578. Other screens can rely on proliferative advantage rather than survival advantage, see, e.g., Jacobs et al. (2000) Nature 397:164-168.

Non-Lethal Selection

Several screening methods which do not rely on proliferation or survival can also be used. For example, screening methods are known which rely upon inducible constructs as surrogates of activated signaling pathways. A signal specific promoter can be used which is usually involved with the activation of a cell surface marker (e.g., CD2) or the promoter can activate expression of a marker, e.g., a fluorescent marker (e.g., GFP or variants thereof). Cells containing a candidate insert nucleic acid which activates expression of the marker can then be isolated, e.g., by fluorescence-activated cell sorting (FACS). Alternatively, a drug resistance marker can be associated with the signal specific promoter. Thus, when a candidate nucleic acid activates expression of the drug resistant marker, indirect lethal selection can be used, i.e., those cells that survive in the presence of the drug can be selected.

In one embodiment, the drug resistant marker can be a marker that allows for both negative and positive selection. For example, a guanine phosphoribosyltransferase encoding sequence or a hygromycin resistance-thymidine kinase fusion encoding sequence can be used. Dual drug markers allow for both positive lethal selection in phenotypic screens and negative selection for the generation of mutant target cells that are defective in a specific signaling cascade. Using an inducible construct, wild-type cells can undergo mutagenesis (e.g., with ICR-191 (see, e.g., Pellegrini et al. (1989) Mol Cell Biol. 9:4605-4612) or EMS (see, e.g., Loh et al. (1992) EMBO J. 11:1351-1363; McKendry et al. (1991) Prot. Natl Acad. Sci. USA 88:11455-11459) and then be negatively selected against the specific signaling process that activates the inducible construct. This can be used to yield mutant cells that can no longer activate the specific signal. Such mutant cells can then be used in a complementation screen with lethal selection to isolate candidate nucleic acids that can correct the defect. For example, as described in Downing et al. (1999) Br. J. Haematol. 106:296-308, ETO-responsive elements have been used in constructs to examine ETO-mediated transcriptional activation. These ETO-response element-containing constructs can drive, e.g., drug resistance markers or GFP proteins. The introduction of nucleic acid libraries then allow for isolation of candidate nucleic acids that upregulate or downregulate ETO transcriptional activity.

Recessive/Suppression Screens

Another method for screening candidate nucleic acids inserted into the vectors described herein includes the generation of inhibitors that inhibit a protein function, thereby mimicking loss of function phenotypes. For these recessive/suppressor screens at least two different approaches can be used to identify candidate insert nucleic acids which inhibit function of a mammalian gene. These include the use of antisense/genetic suppressor elements (GSE) and the use of random peptide libraries, both of which are described below.

Antisense/GSE Screening Methods

The vectors described herein can be used in recessive/suppressor screens to identify candidate nucleic acids through overexpression of full-length or fragment antisense sequences. These screens can, for example, be used to examine the role of a gene in the loss of a cellular function: by providing a phenotype or by providing the cell with a survival and/or proliferative advantage.

Accordingly, the vectors described herein can include a genetic suppressor element (GSE) or full-length antisense sequence. The vector can include a GSE. Such GSE-containing vectors facilitate expression of antisense nucleic acid sequences in mammalian cells. Thus, such GSE-containing vectors can be used, e.g., in conjunction with antisense-based gene inactivation methods. In one embodiment, the GSE-producing vectors further includes one or more of: a packaging sequence (e.g., a packaging sequence having at least one ATG codon which is altered to reduce the formation of fusion polypeptides from the packaging sequence and the insert sequence); a 3′ LTR (e.g., a 3′ LTR which includes a proviral recovery sequence); a 5′ UTR (e.g., a 5′UTR which includes a proviral recovery sequence); an origin of replication; a bacterial selectable marker; and a mammalian selectable marker. In one embodiment, the GSE, the packaging sequence, the origin of replication, the bacterial selectable marker and/or the mammalian selectable marker are located between a 5′LTR and a 3′ LTR.

In one embodiment, antisense genetic suppressor element (GSE)-based methods for the functional inactivation of specific essential or non-essential mammalian genes can be used. Such methods include methods for the identification and isolation of nucleic acid sequences which inhibit the function of a mammalian gene. The methods include those that directly assess a gene's function, as well as those that do not rely on direct selection of a gene's function. These latter methods can be used to identify sequences which affect gene function even in the absence of knowledge regarding such function, e.g., in instances where the phenotype of a loss-of-function mutation within the gene is unknown. An inhibition of gene function, as referred to herein, refers to a reduction in gene expression in the presence of a GSE, relative to the gene's expression in the absence of such a GSE. In one embodiment, the inhibition abolishes the gene's activity, but can be either a qualitative or a quantitative inhibition.

The present invention includes antisense/GSE methods for gene cloning which are based on the function of the gene to be cloned. Such methods can include a method for identifying new nucleic acid sequences based upon the observation that the loss of an unknown gene produces a particular phenotype. The method can include, for example, (a) infecting a cell with a vector described herein having a GSE-containing insert nucleic acid sequence, wherein, upon infection, an integrated provirus is formed and the insert nucleic acid is expressed; and (b) assaying the infected cell for a change in the phenotype, so that new nucleic acid sequences may be isolated based upon the observation that loss of an unknown gene produces a particular phenotype. Such an assay is the same as a sense expression complementation screen except that the phenotype, in this case, is presented only upon loss of function.

In an alternative embodiment, such a method can include a method for identifying a nucleic acid which influences a mammalian cellular function, and can comprise, for example, (a) infecting a cell exhibiting a phenotype dependent upon the function of interest with a vector described herein having a GSE-containing insert nucleic acid sequence, wherein, upon infection, an integrated provirus is formed and the insert nucleic acid is expressed; and (b) assaying the infected cell for the phenotype, so that if the phenotype is suppressed, the insert nucleic acid represents a nucleic acid which influences the mammalian cellular function. For example, a GSE library or full length antisense library can be used as insert nucleic acids in the vectors described herein in order to screen for genes involved in drug sensitivity, radiation sensitivity, or cytokine sensitivity (e.g., IFNγ or TGF-β sensitivity). See, e.g., Carnero et al. (2000) Nucl. Acid Res. 28(11): 2234-2241; Kissil et al. (1995) J. Biol. Chem. 270(46): 27932-27936; Gudkov et al. (1994) Genetics 91:3744-3748. Such assays are the same as a sense expression complementation screen except that the phenotype, in this case, is presented only upon loss of function.

In other aspects, the screening methods can be used to identify a GSE or a different type of suppressor element, e.g., double stranded RNA, that is capable of inhibiting a gene of interest. For example, in one embodiment, the vector can include both a candidate GSE and a nucleic acid sequence comprising at least part of a gene of interest. In other embodiments, a cell expressing the nucleic acid comprising at least a portion of the gene of interest can be infected with a candidate GSE-containing vector described herein. Such a method for identifying an insert nucleic acid sequence which inhibits the function of a mammalian gene of interest can include (a) infecting a mammalian cell with a vector described herein which includes a candidate GSE and a nucleic acid sequence from the gene of interest or infecting a cell which expresses the nucleic acid of interest with a candidate GSE-containing vector described herein. The nucleic acid of interest can encode a fusion protein, e.g., such that the N-terminal portion of the sequence encodes at least a portion of the amino acid sequence of the gene and the C-terminal portion encodes a selectable marker (e.g., a quantifiable marker). The integrated retroviral provirus can then be produced which expresses the candidate GSE nucleic acid (and optionally, nucleic acid sequence of the gene of interest); (b) the selectable marker can be selected for; and (c) the quantifiable or selectable marker can be assayed, so that if the selectable marker is inhibited, a nucleic acid sequence (GSE) which inhibits the function of the mammalian gene is identified.

In one preferred embodiment of this identification method, the fusion protein is encoded by a nucleic acid whose transcription is controlled by an inducible regulatory sequence so that expression of the fusion protein is conditional. In another embodiment of the identification method, the mammalian cell is derived from a first mammalian species and the gene is derived from a second species, a different species as distantly related as is practical.

In a fusion protein-independent embodiment, the nucleic acid encoding the selectable marker can be inserted into the gene of interest such that the selectable marker is translated instead of the gene of interest. This embodiment is useful, for example, in instances in which a fusion protein may be deleterious to the cell in which it is to be expressed, or when a fusion protein cannot be made. The method for identifying a nucleic acid sequence which inhibits the function of a mammalian gene, in this instance, can comprise: (a) infecting a mammalian cell expressing the sequence derived from the gene of interest (e.g., a regulatory sequence of a gene of interest and a sequence encoding a selectable marker) with a vector described herein containing a candidate GSE or by infecting a mammalian cell with a vector described herein containing a candidate GSE and a nucleic acid sequence derived from the gene of interest (e.g., a regulatory sequence of a gene of interest and a sequence encoding a selectable marker). Upon infection, an integrated provirus is formed and the candidate GSE nucleic acid sequence is expressed; (b) selecting for the selectable marker; and (c) assaying for the selectable marker, so that if the selectable marker is inhibited, a nucleic acid sequence (GSE) which inhibits the function of the mammalian gene is identified. Selection for the marker should be quantitative, e.g., by FACS.

In an additional embodiment, the gene of interest and the selectable marker can be placed in operative association with each other within a bicistronic message cassette, separated by an internal ribosome entry site, whereby a single transcript is produced encoding, from 5′ to 3′, the gene product of interest and then the selectable marker. The sequence within the bicistronic message derived from the gene of interest can include not only coding, but also 5′ and 3′ untranslated sequences. The method for identifying a nucleic acid sequence which inhibits the function of a mammalian gene, in this instance, can comprise: (a) infecting a mammalian cell expressing a selectable marker as part of such a bicistronic message with a candidate GSE-producing retroviral vector (e.g., a vector also containing a nucleic acid sequence derived from the gene of interest), wherein, such infection, an integrated provirus is formed and the candidate GSE nucleic acid sequence is expressed; (b) selecting for the selectable marker; and (c) assaying for the selectable marker, so that if the selectable marker is inhibited, a nucleic acid sequence (GSE) which inhibits the function of the mammalian gene is identified.

Nucleic acid sequences identified via such methods can be utilized to produce a functional knockout of the mammalian gene. A “functional knock-out”, as used herein, refers to a situation in which the GSE acts to inhibit the function of the gene of interest, and can be used to refer to a functional knockout cell or transgenic animal.

The present invention also includes methods for the isolation of nucleic acid molecules identified via the antisense or GSE screening methods of the invention. Such methods can utilize PCR-mediated rescue or the proviral recovery sequences in the restriction enzyme mediated or Cre-mediated methods as described herein.

Screens Using Random Peptide Libraries

The vectors described herein can be used for the display of constrained and unconstrained random peptide sequences as part of the insert nucleic acid. Such vectors are designed to facilitate the selection and identification of random peptide sequences that bind to a protein of interest or interrupt protein signaling. The random peptide fragment can be about 5 to 100, about 10 to 50, about 20 to 40 amino acids in length.

Vectors displaying random peptide sequences can include one or more of: a splice donor site or a LoxP site (e.g., LoxP511 site); a bacterial promoter (e.g., pTac) and a shine-delgarno sequence; a pel B secretion signal for targeting fusion peptides to the periplasm; a splice-acceptor site or another LoxP511 site (LoxP511 sites will recombine with each other, but not with the LoxP site in the 3′LTR); a peptide display cassette or vehicle; an amber stop codon; the M13 bacteriophage gene 111 protein C-terminus (e.g., amino acids 198-406); or a linker, e.g., a polyglycine linker.

In one embodiment, the insert nucleic acid includes a peptide display cassette and the peptide display cassette includes a vector polypeptide, e.g., a natural or synthetic polypeptide, into which a polylinker has been inserted into one flexible loop of the natural or synthetic protein. A library of random oligonucleotides encoding random peptides may be inserted into the polylinker, so that the peptides are expressed as part of the vector polypeptide. The vector polypeptide can be, e.g., thioredoxin, and can be used for intracellular peptide display in mammalian cells (See, e.g., Colas et al. (1996) Nature 380:548-550). In an alternative embodiment, the vector polypeptide can be for extracellular peptide display in mammalian cells. For example, the vector polypeptide can be a minibody (See, e.g., Tramonteno (1994) J. Mol. Recognit. 7:9-24) preceded by a secretion signal and followed by a membrane anchor, such as the one encoded by the last 37 amino acids of DAF-1 (Rice et al. (1992) Proc. Natl. Acad. Sci. USA 89:5467-5471). The extracellular display cassette can be flanked by recombinase sites (e.g., frt sites) to allow the production of secreted proteins following passage of the library through a recombinase expressing host.

In an amber suppressor strain of bacteria and in the presence of helper phage, these vectors would produce a relatively conventional phage display library which could be used exactly as has been previously described for conventional phage display vectors. Recovered phage that display affinity for the selected target would be used to infect bacterial hosts of the appropriate genotype (i.e., expressing the desired recombinases depending upon the cassettes that must be removed for a particular application). For example, for intracellular peptide display, any bacterial host would be appropriate (provided that splice sites are used to remove pelB in the mammalian host). For secreted peptide display, the minibody vector can be passed through bacterial cells that catalyze the removal of the DAF anchor sequence. Plasmids prepared from these bacterial hosts can be used to produce virus particles for assaying specific phenotypes in mammalian cells.

In some cases, if the target is unknown, the phage display step could be skipped and the vectors could be used for intracellular or extracellular random peptide display directly. The advantage of these vectors over conventional approaches is their flexibility. The ability to functionally test the peptide sequence in mammalian cells without additional cloning or sequencing steps makes possible the use of much cruder binding targets (e.g., whole fixed cells) for phage display. This is made possible by the ability to do a rapid functional selection on the enriched pool of bound phages by conversion to retroviruses that can infect mammalian cells.

Methods of Screening for Genes Modulated in Response to a Stimuli

The present invention further relates to gene trapping-based methods for the identification and isolation of mammalian genes which are modulated in response to specific stimuli. These methods utilize retroviral particles of the invention to infect cells, which leads to the production of provirus sequences which are randomly integrated within the recipient mammalian cell genome. In instances in which the integration event occurs within a gene, the gene is “tagged” by the provirus reporter sequence, whose expression is controlled by the gene's regulatory sequences. By assaying reporter sequence expression, then, the expression of the gene itself can be monitored.

In one embodiment, the reporter sequence encodes a quantifiable selectable marker that can be assessed, e.g., by FACS analysis. This allows for the isolation of clones that are either induced or repressed.

The term “modulation”, as used herein, refers to an up- or down-regulation of gene expression in response to a specific stimulus in a cell. The modulation can be either a quantitative or a qualitative one.

The selection method can include, for example: (a) infecting a mammalian cell with a retrovirus derived from a vector described herein, wherein, upon infection, an integrated provirus is formed; (b) subjecting the cell to the stimulus of interest; and (c) assaying the cell for the expression of the reporter sequence such that if the reporter sequence is expressed, it is integrated within, and thereby identifies, a gene that is expressed in the presence of the stimulus. When the gene is not expressed or, alternatively, is expressed at a different level, in the absence of the stimulus, the method identifies a gene which is expressed in response to a specific stimulus.

The present invention also includes methods for the isolation of nucleic acid sequence expressed in the presence of, or expressionally responsive to, a specific stimulus. Such methods can include, for example, digesting the genome of a cell which contains a provirus integrated into a gene which is expressed in the presence of, or in response to, the stimulus of interest; and recovering a nucleic acid containing a sequence of the gene by utilizing the means for recovering nucleic acid sequences from a complex mixture of nucleic acid.

Such methods serve to recover proviral nucleic acid sequence along with flanking genomic sequence (i.e., sequence contained within the gene of interest). The isolated sequence can be circularized, yielding a plasmid capable of replication in bacteria. This is made possible by the presence of a bacterial origin of replication and a bacterial selectable marker within the isolated sequence.

Upon isolation of flanking gene sequence, the sequence can be used in connection with standard cloning techniques to isolate nucleic acid sequences corresponding to the full length gene of interest. See, e.g., U.S. Pat. No. 6,025,192, the contents of which are incorporated herein by reference.

In another embodiment, the methods can be used to identify a target nucleic acid encoding a polypeptide which causes a desired change in a cellular phenotype, e.g., a change in a cellular phenotype that is associates with a disease. The methods utilize retroviral particles of the invention to introduce a library of random peptide or protein probes into a group of cells of a cell-type of interest, which leads to the production of provirus sequences which are integrated within recipient cell genomes. Each cell of the cell-type of interest can have a different sequence encoding a different peptide probe. Once in the cell, the peptide probe can be expressed and can interact with different potential targets within the cell. In one embodiment, the peptide can be expressed in a specific location within the cell, e.g., the cytoplasm and/or nucleus. The cell can then be subjected to a stimulus of interest, e.g., a stimulus which results in the cell displaying a phenotype, e.g., a phenotype associated with a disease. The cells can then be assayed to identify cells which do not display the phenotype of the disease, preferably without causing other undesirable phenotypic changes in the cell. For example, proviral sequences encoding various peptide probes can be introduced into a mast cell. The mast cell can then be subjected to a stimulus which normally results in histamine release from wild-type mast cells. Those cells which do not release histamine can be identified. Such methods are described, for example, U.S. Pat. No. 6,153,380, the contents of which is incorporated herein by reference.

The present invention also includes methods for the isolation of a nucleic acid sequence expressed in the presence of, or in response to, a specific stimulus. Such methods can include, for example, digesting the genome of a cell which contains a provirus integrated into a gene which is expressed in the presence of, or in response to, the stimulus of interest; and recovering a nucleic acid containing a sequence of the gene by utilizing the means for recovering nucleic acid sequences from a complex mixture of nucleic acid.

Such methods serve to recover proviral nucleic acid sequence along with flanking genomic sequence (i.e., sequence contained within the gene of interest). The isolated sequence can be circularized, yielding a plasmid capable of replication in bacteria. This is made possible by the presence of a bacterial origin of replication and a bacterial selectable marker within the isolated sequence.

Mutant Allele Identification

Finally, the pEYK vector systems can be utilized to identify mutant alleles of a specific gene. The desired gene can be altered through mutagenic PCR conditions or through mutagenic bacterial strains. Such methods are adaptable for rapid screening of the gene libraries generated by combinatorial mutagenesis of the sequence of interest. Recursive ensemble mutagenesis (REM), a new technique which enhances the frequency of functional mutants in the libraries, can be used in combination with the screening assays to identify variants (Arkin and Yourvan (1992) Proc. Natl. Acad. Sci. USA 89:7811-7815; Delgrave et al. (1993) Protein Engineering 6:327-331). Such strategies have identified oncogenic forms of the c-mpl (thrombopoietin receptor) gene (Onishi et al. (1996) Blood 88:1399-1406) or constitutively active forms of the STAT5 gene (Ariyoshi et al. (2000); Onishi et al. (1998) Mol. Cell Biol. 18:3871-3879). These mutagenic screens can be used to identifying alleles that are neomorphs or dominant-negatives-both useful reagents in understanding gene function.

ORF Expression Libraries

With the almost-complete identification of all the human opening reading frames (ORFs), the genomic effort has turned to generating expression libraries that contain all of the full-length ORFs in expression vector. For example, Life Technologies has generated the GatewayTM system (www.lifetech.com) to facilitate moving full-length ORFs using recombinase enzymes into expression vectors. All the GatewayTM expression vectors currently are transient expression shuttle vectors, which cannot be used in phenotypic screens that require stable expression. By adapting the GatewayTM system to the pEYK retroviral vectors, such a system can allow phenotypic screens using the majority of full-length ORFs. In addition, all the cDNAs can be represented in full-length form, making the screen significantly more efficient. Instead of screening millions of infected cells, one would only need to screen around 100,000 infected cells to saturate the complexity of ORF library.

The final step will be to spatially segregate expression libraries on glass arrays, bypassing the need to recover and identify the clone. A preliminary technique—reverse transfection—uses spatially segregated transient expression constructs on glass slides and has validated the idea of in vivo expression chips. Unfortunately, reverse transfection can only confer transient expression of the ORF/cDNA and depends on the limitation of transfection techniques, thereby limiting the range of target cells that can be screened. The solution may require the use of spatial segregation to generate small volumes of retroviral supernatants for subsequent small-scale infections to maximize the depths of the phenotypic screens.

Recombinational Cloning

Methods for recombinational cloning are well known in the art (see e.g., U.S. Pat. No. 5,888,732; Walhout et al. (2000) Science 287:116; Liu et al. (1998) Curr. Biol. 8(24): 1300-9.). Recombinational cloning exploits the activity of certain enzymes that cleave DNA at specific sequences and then rejoin the ends with other matching sequences during a single concerted reaction.

U.S. Pat. No. 5,888,732 describes a system based upon the site-specific recombination of bacteriophage lambda and uses double recombination. In double recombination, any DNA fragment that resides between the two different recombination sites will be transferred to a second vector that has the corresponding complementary sites. The system relies on two vectors, a master clone vector and a target vector. The one harboring the original gene is known as the master clone. The second plasmid is the target vector, the vector required for a specific application, such as a vector described herein for programming an array. Different versions of the expression vectors are designed for different applications, e.g., with different affinity and/or recognition tags, but all can receive the gene from the master clone. Site-specific recombination sites are located within the expression vector at a location appropriate to receive the coding nucleic acid sequence harbored in the master clone. To shuttle the gene into the target vector, the master clone vector containing a nucleic acid sequence of interest and the target vector are mixed with the recombinase.

The mixture is transformed into an appropriate bacterial host strain. The master clone vector and the target vector can contain different antibiotic selection markers. Moreover, the target vector can contain a gene that is toxic to bacteria that is located between the recombination sites such that excision of the toxic gene is required during recombination. Thus, the cloning products that are viable in bacteria under the appropriate selection are almost exclusively the desired construct. In practice, the efficiency of cloning the desired product approaches 100%.

Each gene is amplified from an appropriate cDNA library using PCR. The recombination sequences are incorporated into the PCR primers so the amplification product can be directly recombined into a master vector. As described above, because the master vector carries a toxic gene that is lost only after successful recombination, the desired master clone is the only viable product of the process. Once in the master vector, the gene can be verified, e.g., by sequencing methods, and then shuttled into any of the many available expression vectors.

Because of the ease of shuttling multiple genes to any expression vector en masse, these clones can be prepared to construct libraries, such as those described herein.

Liu et al. (1998) Curr. Biol. 8:1300 describe a Cre-lox based site-specific recombination system for the directional cloning of PCR products. This system uses Cre-Lox recombination and a single recombination site. Here again the master clone is mixed with a target vector and recombinases. However, instead of swapping fragments, the recombination product is a double plasmid connected at the recombination site. This then juxtaposes one end of the gene (whichever end was near the recombination site) with the desired signals in the expression plasmid.

The clone can include a vector sequence described herein and a full-length coding region of interest. The coding region can be flanked by marker sequences for site-specific recombinational cloning, e.g., Cre-Lox sites, or lambda int sites (see, e.g., Uetz et al. (2000) Nature 403:623-7). Also, the coding region can be flanked by marker sequences for homologous recombination (see, e.g., Martzen et al. (1999) Science 286:1153-5). For homologous recombination almost any sequence can be used that is present in the vector and appended to the coding region.

Exemplary Useful Sequences

Naturally Occurring Sequences

Useful encoding nucleic acid sequence for creating libraries include naturally occurring sequences. Nucleic acid sequences can be procured from cells of species from the kingdoms of animals, bacteria, archebacteria, plants, and fungi. Non-limiting examples of eukaryotic species include: mammals such as human, mouse (Mus musculus), and rat; insects such as Drosophila melanogaster. In addition, amino acid sequence encoded by viral genomes can be used, e.g., a sequence from rotavirus, hepatitis A virus, hepatitis B virus, hepatitis C virus, herpes virus, papilloma virus, or a retrovirus (e.g., HIV-1, HIV-2, HTLV, SIV, and STLV).

In one embodiment, a cDNA library is prepared from a desired tissue of a desired species and is inserted in a vector described herein.

Artificial Sequences

The encoding nucleic acid sequence can encode artificial amino acid sequences. Artificial sequences can be randomized amino acid sequences, patterned amino acid sequence, computer-designed amino acid sequences (see, e.g., Dahiyat and Mayo (1997) Science 278:82-7), and combinations of the above with each other or with naturally occurring sequences. Cho et al. (2000) J Mol Biol 297:309-19 describes methods for preparing libraries of randomized and patterned amino acid sequences. Similar techniques using randomized oligonucleotides can be used to construct libraries of random sequences. Individual sequences in the library (or pools thereof) can be inserted.

The encoding sequences can also encode a naturally occurring polypeptide which is modified in part to express an artificial peptide sequence, e.g., an epitope. Norman et al. (1999) Science 285:591-5 described a method of displaying functional regions on an RnaseA scaffold protein in order to alter cellular functions. Methods of generating nucleic acids encoding such sequences include mutagenesis methods described below.

Mutagenesis

The library can be used to express the products of a mutagenesis or selection. Examples of mutagenesis procedures include cassette mutagenesis (see e.g., Reidhaar-Olson and Sauer (1988) Science 241:53-7), PCR mutagenesis (e.g., using manganese to decrease polymerase fidelity), in vivo mutagenesis (e.g., by transfer of the nucleic acid in a repair deficient host cell), and DNA shuffling (see U.S. Pat. Nos. 5,605,793; 5,830,721; and 6,132,970). Examples of selection procedures include complementation screens, and phage display screens

In addition, more methodical variation can be achieved. For example, an amino acid position or positions of a naturally occurring protein can be systematically varied, such that each possible substitution is present at a unique position. For example, the all the residues of a binding interface can be varied to all possible other combinations. Alternatively, the range of variation can be restricted to reasonable or limited amino acid sets.

Collections

Additional collections include libraries having at different addresses one of the following combinations: combinatorial variants of a bioactive peptide; specific variants of a single polypeptide species (splice variants, isolated domains, domain deletions, point mutants); polypeptide orthologs from different species; polypeptide components of a cellular pathway (e.g., a signalling pathway, a regulatory pathway, or a metabolic pathway); and the entire polypeptide complement of an organism.

Repositories of Nucleic Acids

The library described herein can be produced by cloning of individual member of a collection of nucleic acid sequences. Such a collection can be obtained, e.g., from a supplier of isolated nucleic acid clones, e.g., full length cDNAs from human and other mammalian organisms to make a library of this size.

The clones in the collection can be maintained, produced, or obtained in a format compatible with recombination-mediated cloning, e.g., as described above. Such a methodology is reliable for high throughput shuttling of insert sequences into a vector, e.g., a vector nucleic acid described herein, and can reduce the number of library clones that are required to be screened to obtain reasonable coverage of a collection. Such a collection can be used to produce pseudotyped viral particles containing the nucleic acids of interest. The collection can be screened in cells, as described herein.

All patents and other references cited herein are hereby incorporated by reference.

Other embodiments are within the following claims.

What is claimed:

1. A vector comprising from 5′ to 3′:
a) a packaging sequence;
b) a heterologous insert sequence or restriction sites for insertion of a heterologous sequence; and
c) a 3′ long terminal repeat (LTR) sequence,
wherein at least two codons of the packaging sequence are altered so as to reduce formation of fusion polypeptides encoded by the packaging sequence or a portion thereof, and the heterologous insert sequence.

2. The vector of claim 1, wherein at least two ATG codons of the packaging sequence have been altered.

3. The vector of claim 2, wherein the ATG initiation codon of the packaging sequence and at least one internal ATG codon of the packaging sequence have been altered.

4. The vector of claim 1, wherein the packaging sequence is a gag sequence.

5. The vector of claim 4, wherein the gag sequence is an amino-terminal portion of the gag gene.

6. The vector of claim 4, wherein the gag sequence comprises the nucleotide sequence of SEQ ID NO:2, or a portion thereof

7. The vector of claim 3, wherein at least two internal ATG codons of the packaging sequence have been altered.

8. The vector of claim 3, wherein the internal codon which is altered is the codon at residues 1097-1099 of SEQ ID NO:1.

9. The vector of claim 3, wherein the internal codon which is altered is the codon at residues 1589-1591 of SEQ ID NO:1.

10. The vector of claim 3, wherein the internal codon at residues 1097-1099 and the internal codon at residues 1589-1591 of SEQ ID NO:1 have been altered.

11. The vector of claim 2, wherein one, two or all of the nucleotides of the ATG codon(s) have been altered.

12. The vector of claim 1, wherein the vector includes a heterologous insert sequence.

13. A vector comprising from 5′ to 3′:
a) a packaging sequence, wherein at least one ATG codon of the packaging sequence has been altered;
b) a heterologous insert sequence or restriction sites for insertion of a heterologous sequence; and
c) a 3′ LTR sequence, wherein the 3′ LTR comprises a proviral recovery sequence.

14. The vector of claim 13, wherein at least two ATG codons of the packaging sequence have been altered.

15. The vector of claim 14, wherein the ATG initiation codon of the packaging sequence and at least one internal ATG codon of the packaging sequence have been altered.

16. The vector of claim 13, wherein the packaging sequence is a gag sequence.

17. The vector of claim 16, wherein the gag sequence is an amino-terminal portion of the gag gene.

18. The vector of claim 14, wherein at least two internal ATG codons of the packaging sequence have been altered.

19. The vector of claim 14, wherein the internal codon which is altered is the codon at residues 1097-1099 of SEQ ID NO:1.

20. The vector of claim 14, wherein the internal codon which is altered is the codon at residues 1589-1591 of SEQ ID NO:1.

21. The vector of claim 14, wherein the internal codon at residues 1097-1099 and the internal codon at residues 1589-1591 of SEQ ID NO:1 have been altered.

22. The vector of claim 14, wherein all of the nucleotides of the ATG codon(s) have been altered.

23. The vector of claim 13, wherein the vector includes a heterologous insert sequence.

24. The viral vector of claim 13, further comprising a bacterial origin of replication.

25. The viral vector of claim 24, wherein at least a portion of the bacterial origin of replication has been removed.

26. The viral vector of claim 13, wherein the bacterial marker sequence is a bleomycin marker sequence.

27. The viral vector of claim 13, wherein the proviral recovery sequence is located within a portion of the 3′ LTR which duplicates upon integration.

28. A vector comprising from 5′ to 3′:
a) a packaging sequence, wherein at least one ATG codon of the packaging sequence has been altered;
b) a heterologous insert sequence or restriction sites for insertion of a heterologous sequence;
c) a bacterial marker sequence, wherein the bacterial marker is less than 600 basepairs in length; and
d) a 3′ LTR sequence, wherein the 3′ LTR comprises a proviral recovery sequence.

29. A viral vector comprising:
a) a packaging sequence;
b) a heterologous insert sequence;
c) a bacterial marker sequence, wherein the bacterial marker sequence is less than 600 basepairs in length;
d) a 3′ LTR comprising a proviral recovery sequence,
wherein the vector comprises and can express a heterologous insert sequence greater than about 8 kilobases in length.

30. The viral vector of claim 29, wherein the packaging sequence is altered at an initiation codon of the packaging sequence and at least one potential initiation codon of the packaging sequence.

31. The viral vector of claim 29, further comprising a bacterial origin of replication.

32. The viral vector of claim 31, wherein at least a portion of the bacterial origin of replication has been removed.

33. The viral vector of claim 29, wherein the bacterial marker sequence is a bleomycin marker sequence.


SEQUENCE LISTING (15 sequences)

SEQ ID NO: 1 - DNA, 5782 bp, Artificial Sequence
  Synthetically generated nucleic acid
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaagcggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccaggtcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatcccagtgtggtggtagggaattcaagcttgatctctataatctcgcgcaacctattttcccctcgaacactttttaagccgtagataaacaggctgggacacttcacatgagcgaaaaatacatcgtcacctgggacatgttgacagatccatgcacgtaaactcgcaagccgactgatgccttctgaacaatggaaaggcattattgccgtaagccgtggcggtctggtaccggtgggtgaagaccagaaacagcacctcgatctgagccgcgatattgcccagcgtttcaacgcgctgtatggcgagatcgatcccgtcgttttacaacgtcgtgactgggaaaaccctggcgttacccaacttaatggccttggaggacatccccctttcgccagctggcgtaatagcgaagaggcccgcaccgatcgcccttcccaacagttgcgcagcctgaattggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacattttaatgttgatgaaagctggctacaggaaggccagacgcgaattatttttgatggcgttaactcggcgtttcatctgtggtgcaacgggcgctgggtcggttacgggcaagacagtcgtttggcgtcttaatttgagctcgagcgcatatctacgcgccggagaaaaccgcctcgcggtgatggtgctgcgctggagtgacgggagttatcttgaagatcaagatatgtggcggatgagcgggattccgagcgaaaacggtctgcgctgcgggacgcgcgaattgaattatggcccacaccagagtgggcgcggcgacttccagttcaacatcagccgctacagtcaacagcaactgatggaaaccagccatcgccatctgctgcacgcggaagaaccgacatggctgttatacgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaactcgagcggccgccagcacagtggtcgacgataaaataaaagattttatttagtctccagaaaaaggggggaatgaaagaccccacctgtaggtttggcaagctagcttaagtaacgccattttggaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctgcat

SEQ ID NO: 2 - DNA, 4141 bp, Artificial Sequence
  Synthetically generated nucleic acid
aatgaaagaccccacctgtaggtttggcaagctagcgcggccgcataacttcgtatagcatacattatacgaagttatttaattaaggcgcgcctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaataagggcccgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacaagacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatcccagtgtggtggtagggaattcttaattaacgccaccatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaattaattaagaattccagctgagcgccggtcgctaccattaccagttggtctggtgtcaaaaataataataaccgggcaggccatgtctgcccgtatttcgcgtaaggaaatccattatgtactatttaaactcgagcggccggccgccagcacagtggtcgactgttgacaattaatcatcggcatagtatatcggcatagtataatacgacaaggtgaggaactaaaccatggccaagttgaccagtgccgttccggtgctcaccgcgcgcgacgtcgccggagcggtcgagttctggacccgaccggctcgggttctcccgggacttcgtggaggacgacttcgcccggtgtggtccgggacgacgtgactctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaacgcgtcccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatatcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 3 - DNA, 25 bp, Artificial Sequence
  Synthetically generated primer
aaaggaccttacacagtcctgctga

SEQ ID NO: 4 - DNA, 25 bp, Artificial Sequence
  Synthetically generated primer
caccacaggtaatgcttttactggc

SEQ ID NO: 5 - DNA, 25 bp, Artificial Sequence
  Synthetically generated primer
gaccttacacagtcctgctgaccac

SEQ ID NO: 6 - DNA, 25 bp, Artificial Sequence
  Synthetically generated primer
aagaacctagaacctcgctggaaag

SEQ ID NO: 7 - DNA, 25 bp, Artificial Sequence
  Synthetically generated primer
gaagtcgatgacggcagatttagag

SEQ ID NO: 8 - DNA, 25 bp, Artificial Sequence
  Synthetically generated primer
ccacaggtaatgcttttactggcct

SEQ ID NO: 9 - DNA, 25 bp, Artificial Sequence
  Synthetically generated primer
aaggaccttacacagtcctgctgac

SEQ ID NO: 10 - DNA, 25 bp, Artificial Sequence
  Synthetically generated primer
gccgacaccagactaagaacctaga

SEQ ID NO: 11 - DNA, 25 bp, Artificial Sequence
  Synthetically generated primer
aaccccaccacaggtaatgctttta

SEQ ID NO: 12 - DNA, 25 bp, Artificial Sequence
  Synthetically generated primer
acaggtaatgcttttactggcctgc

SEQ ID NO: 13 - DNA, 25 bp, Artificial Sequence
  Synthetically generated primer
gccgctgtaaagtgttacgttgaga

SEQ ID NO: 14 - DNA, 10 bp, Artificial Sequence
  Synthetically generated oligonucleotide
aaatatgggc

SEQ ID NO: 15 - DNA, 10 bp, Artificial Sequence
  Synthetically generated oligonucleotide
aaataagggc